(19) United States
(12) Patent Application Publication
(10) Pub. No.: US 2024/0206902 A1
(43) Pub. Date: Jun. 27, 2024

(54) PULMONARY ARTERIAL HYPERTENSION CATHETERS

(71) Applicant: NXT Biomedical, LLC, Irvine, CA (US)

(72) Inventors: Robert C. Taft, Irvine, CA (US); Elliot Howard, Irvine, CA (US); Alexander Siegel, Irvine, CA (US); Glen Rabito, Irvine, CA (US)

(73) Assignee: NXT Biomedical, LLC, Irvine, CA (US)

(21) Appl. No.: 18/398,105

(22) Filed: Dec. 27, 2023

Related U.S. Application Data
(60) Provisional application No. 63/477,345, filed on Dec. 27, 2022.

(51) Int. Cl. A61B 17/32; A61B 8/12; A61B 17/12

(52) U.S. Cl. CPC A61B 17/320016; A61B 8/12; A61B 17/1204; A61B 17/12109; A61B 17/12136; A61B 2017/1107; A61B 2017/320056

(57) ABSTRACT

Disclosed herein are devices and methods for creating a shunt between two vessels or lumens within a patient. While these devices and methods are generally described with regard to treatment of hypertension (e.g., pulmonary arterial hypertension) and/or right heart failure/disfunction, they can be used with a variety of different vessels and lumens for other purposes. The devices include puncturing guidewire embodiments that can more accurately pierce two vessels, as well as snare catheter designs that can prevent unwanted damage from a puncturing guidewire.

### RELATED APPLICATIONS

This application claims benefit of and priority to U.S. Provisional Application Ser. No. 63/477,345 filed Dec. 27, 2022 entitled Rivet Stent Puncture System And Methods, which is hereby incorporated herein by reference in its entirety.

### BACKGROUND OF THE INVENTION

Pulmonary hypertension is a condition that describes high blood pressure in the lungs. There are a variety of causes for the increased pulmonary blood pressure, including obstruction of the small arteries in the lung, high left-sided heart pressures, and chronic lung disease.

There are many medical conditions that also create high pulmonary blood pressure as a secondary condition, including heart failure. In heart failure, the heart is unable to meet the demand for blood coming from the body. This often leads to increased pressures within the heart that can back up into the lungs causing pulmonary hypertension at rest or during exercise.

In almost all cases, this increased pulmonary blood pressure causes the right ventricle to work harder to supply the lungs and the left side of the heart with blood. Over time, this additional load causes damage to the heart, decreasing efficiency and limiting the ability to keep up with the demands of the body, especially during exercise.

Reducing pulmonary blood pressure has been the target of numerous therapies, especially in patients with pulmonary arterial hypertension where several drugs have shown moderate success. However, these drugs are often very expensive and burdensome to the patient and over time can lose their effectiveness.

In this regard, what is needed is an improved treatment option for reducing pulmonary blood pressure and other conditions of elevated blood pressure.

### SUMMARY OF THE INVENTION

Disclosed herein are improved devices and methods for creating a shunt between two vessels or lumens within a patient. While the devices and methods may be particularly useful in creating a shunt between a superior vena cava (SVC) and a right pulmonary artery (RPA), other shunt locations are also possible.

One embodiment is directed to a delivery device catheter configured to deliver a shunt support structure without a sheath over the delivery device while crossing one or more vessel walls. The catheter can include one or more proximal or distal cones that cover only a proximal and/or distal end of the support structure. The cones can be slidable and biased to a position covering the support structure or can be configured to at least partially rip or tear away.

Another embodiment is directed to a delivery device with a distal tip having one or more RF electrodes configured such that the delivery device can pierce one or more vessel walls, dilate one or more vessel walls, and then deliver a shunt support structure to create a shunt between two vessels.

Another embodiment is directed to a radiofrequency piercing guidewire having a biased outer sheath. The outer sheath covers the distal tip of the guidewire in one position and then slides back a predetermined distance to a second position to expose the ablative tip of the guidewire. This may limit the length that the guidewire tip can penetrate beyond a wall of a vessel.

Yet another embodiment is directed to a handle for a radiofrequency piercing guidewire that includes a mechanism (e.g., a thumbwheel or screw drive mechanism) to advance the guidewire out of a sheath a predetermined distance to thereby prevent overextension completely through a vessel wall.

Another embodiment is directed to a snare catheter having an inflatable balloon at its distal tip with one or more snare loops positioned within the balloon, outside of the balloon, or embedded in the balloon material.

Yet another embodiment is directed to a snare catheter having a shield disposed on one side of one or more snare loops. The shield is configured to prevent a piercing guidewire from extending through it.

Another embodiment is directed to a snare catheter having one or more balloons and a shield member. The one or more balloons can be configured to anchor the distal end of the catheter and or center or brace the distal end of the catheter in a desired position. The one or more balloons can be located proximally and/or distally of the shield member.

Yet another embodiment is directed to a snare catheter having one or more perfusion passages. The one or more perfusion passages may extend through one or more balloons or may extend through a body of the catheter.

Another embodiment is directed to a snare catheter with a radiofrequency electrode to help direct radiofrequency current form an RF puncturing guidewire.

Yet another embodiment is directed to a snare catheter having a conductive coil configured to generate a magnetic field. The magnetic field can be used by a puncturing guidewire to sense a position of the conductive coil of the snare and/or to magnetically attract the puncturing guidewire via magnetic force.

Another embodiment is directed to a steerable catheter that includes one or more balloons or expandable rings for positioning and/or bracing a distal end of the catheter. This may allow a puncturing guidewire to more accurately be deployed from the steerable catheter.

Yet another embodiment is directed to a catheter system that includes a catheter sheath having a first lumen for receiving a puncturing system and a deployment system, and a second lumen for receiving an imaging catheter. The imaging catheter may be used to help navigate the catheter system into a position adjacent to a target blood vessel without the use of fluoroscopy.

Another embodiment is directed to a catheter system that includes a reinforced steerable catheter including a slotted or segmented hypotube for reinforcing a distal portion of the catheter. This may allow a puncturing guidewire or crossing catheter to more accurately be deployed from the reinforced steerable catheter.

Yet another embodiment is directed to a catheter system that includes a catheter sheath including a proximal sealing element for temporarily blocking blood flow through a vessel in which the catheter system is located. This may provide for reduced blood pressure during puncturing and/or shunt deployment operations to reduce the risk of internal bleeding between the two vessels.

Yet another embodiment is directed to a catheter system that includes a sealing catheter including a distal sealing element for temporarily blocking blood flow through a vessel in which the catheter system is located. This may provide for reduced blood pressure during puncturing and/or shunt deployment operations to reduce the risk of internal bleeding between the two vessels.

Another embodiment is directed to a catheter system that includes a puncturing guidewire with a helical tip. This may be useable to puncture and prevent lateral movement between two adjacent blood vessels.

Yet another embodiment is directed to a delivery catheter that includes a suture wrap located about a shunt support structure of the delivery catheter. This may help to avoid movement of the shunt support structure along the delivery catheter during advancement of the shunt support structure between two vessels.

Another embodiment is directed to a catheter system including a catheter sheath having a helical or S-shaped profile for contacting and engaging a wall of a vessel in which the catheter system is located to prevent relative movement therebetween.

Yet another embodiment is directed to a catheter assembly that includes an imaging catheter and a lumen for receiving a puncturing system and a deployment system. The imaging catheter may be used to help navigate the catheter system into a position adjacent to a target blood vessel without the use of fluoroscopy.

In some examples, the embodiments described herein relate to a method for creating a shunt, the method including: positioning a catheter sheath within a superior vena cava; inserting an imaging catheter into a lumen extending within the catheter sheath; advancing an imaging catheter out of a distal end of the catheter sheath; imaging a right pulmonary artery with the imaging catheter; anchoring the catheter sheath within the superior vena cava to prevent relative movement between the catheter sheath and the superior vena cava; advancing a puncturing guidewire of a puncturing system out of the distal end of the catheter sheath, through the superior vena cava, and into the right pulmonary artery; advancing a crossing catheter of the puncturing system of the distal end of the catheter sheath, through the superior vena cava, and into the right pulmonary artery; and expanding a shunt support structure within the superior vena cava and the right pulmonary artery to create a shunt passage.

In further aspects, the embodiments described herein relate to a method for creating a shunt, the method including: positioning a catheter sheath within a right pulmonary artery; inserting an imaging catheter into a lumen extending within the catheter sheath; advancing an imaging catheter out of a distal end of the catheter sheath; imaging a superior vena cava with the imaging catheter; anchoring the catheter sheath within the right pulmonary artery to prevent relative movement between the catheter sheath and the right pulmonary artery; advancing a puncturing guidewire of a puncturing system out of the distal end of the catheter sheath, through the right pulmonary artery, and into the superior vena cava; advancing a crossing catheter of the puncturing system out of the distal end of the catheter sheath, through the right pulmonary artery, and into the superior vena cava; and expanding a shunt support structure within the right pulmonary artery and the superior vena cava to create a shunt passage.

In additional aspects, the embodiments described herein relate to a method for creating a shunt, the method including: positioning a catheter sheath within a superior vena cava; inserting an imaging catheter into a lumen extending within the catheter sheath; advancing an imaging catheter out of a distal end of the catheter sheath; imaging a right pulmonary artery with the imaging catheter; anchoring the catheter sheath within the superior vena cava to prevent relative movement between the catheter sheath and the superior vena cava; advancing a puncturing crossing catheter of a puncturing system out of the distal end of the catheter sheath, through the superior vena cava, and into the right pulmonary artery; advancing guidewire of the puncturing system out of the catheter sheath, through the superior vena cava, and into the right pulmonary artery; and expanding a shunt support structure within the superior vena cava and the right pulmonary artery to create a shunt passage.

Yet another embodiment includes one or more combinations of any of the features of the embodiments of this specification, as well as one or more combinations of methods of use of any of the embodiments of this specification.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which:

FIG. 1 is a view of a heart with a diagnostic catheter.

FIG. 2 is a view of a heart with a snare catheter.

FIG. 3 is a view of a vessel with a snare catheter within it.

FIG. 4 is a view of a heart with a snare catheter and a puncture system.

FIG. 5 is a view of a vessel with a snare catheter and a puncture system.

FIG. 6 is a view of a vessel with a snare catheter and a puncture system.

FIG. 7 is a view of a vessel with a snare catheter and a puncture system.

FIG. 8 is a view of a vessel with a snare catheter and a puncture system.

FIG. 9 is a view of a shunt support structure forming a stent between two vessels.

FIG. 10 is a view of a compressed shunt support structure.

FIG. 11 is a view of an expanded shunt support structure.

FIG. 12 is a view of a compressed shunt support structure.

FIG. 13 is a view of an expanded shunt support structure.

FIG. 14 is a view of a shunt support structure delivery catheter.

FIG. 15 is a view of a shunt support structure delivery catheter.

FIG. 16 is a view of a shunt support structure delivery catheter.

FIG. 17 is a view of a shunt support structure delivery catheter.

FIGS. 18, 19, and 20 are views of a puncturing guidewire.

FIGS. 21 and 22 are views of a puncturing guidewire handle.

FIG. 23 is a view of a snare catheter.

FIG. 24 is a view of a snare catheter.

FIG. 25 is a view of a snare catheter.

FIG. 26 is a view of a snare catheter and a puncturing guidewire.

FIG. 27 is a view of a snare catheter and a puncturing guidewire.

FIG. 28 is a view of a snare catheter and a puncturing guidewire.

FIG. 29 is a view of a snare catheter and a puncturing guidewire.

FIG. 30 is a view of a snare catheter and a puncturing guidewire.

FIG. 31 is a view of a snare catheter and a puncturing guidewire.

FIG. 32 is a view of a snare catheter and a puncturing guidewire.

FIG. 33 is a view of a steerable or crossing catheter.

FIG. 34 is a view of a steerable or crossing catheter.

FIG. 35 is a view of a steerable or crossing catheter.

FIG. 36 is a view of a steerable or crossing catheter.

FIG. 37 is a view of steerable or crossing catheter.

FIG. 38 is a view of steerable or crossing catheter.

FIG. 39 is a view of catheter with a side aperture.

FIG. 40 is a view of catheter with a side aperture.

FIG. 41 is a view of catheter with a side aperture.

FIG. 42 is a view of catheter with a side aperture.

FIG. 43 is a view of catheter with a side aperture.

FIG. 44 is a view of catheter with a side aperture.

FIGS. 45, 46, 47, 48, and 49 are views of a catheter with a side aperture and

radiopaque markers.

FIGS. 50, 51, 52, and 53 are views of a catheter with a side aperture and a magnetic connection mechanism.

FIG. 54 is a view of two catheters with a magnetic connection mechanism.

FIGS. 55, 56, and 57 are views of a balloon snare catheter.

FIG. 58 is a cross-section view of a catheter system in a superior vena cava.

FIG. 59 is a view of the catheter system of FIG. 58 in the superior vena cava.

FIG. 60 is view of the catheter system of FIGS. 58-59 in a superior vena cava.

FIG. 61 is a view of a catheter system in a superior vena cava.

FIG. 62 is a view of a catheter system in a superior vena cava.

FIG. 63 is view of a catheter system in a superior vena cava.

FIG. 64 is a view of a catheter system in a superior vena cava.

FIG. 65 is a view of a catheter system in a superior vena cava.

FIG. 66 is a cross-section view of the catheter system of FIG. 65 in a superior vena cava.

FIG. 67 is a view of a catheter system in a superior vena cava.

FIG. 68 is view of a catheter assembly in a superior vena cava.

FIG. 69 is a cross-section view of the catheter assembly in a superior vena cava.

FIG. 70 is a view of a catheter assembly in a superior vena cava.

FIG. 71 is view of a catheter assembly in a superior vena cava.

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements. While different embodiments are described, features of each embodiment can be used interchangeably with other described embodiments. In other words, any of the features of each of the embodiments can be mixed and matched with each other, and embodiments should not necessarily be rigidly interpreted to only include the features shown or described.

Disclosed herein are improved devices and methods for creating a shunt between two vessels or lumens within a patient. While these devices and methods are generally described with regard to treatment of hypertension (e.g., pulmonary arterial hypertension) and/or right heart failure/disfunction, it should be understood that they can be used with a variety of different vessels and lumens for other purposes.

Shunts can be used to connect several different locations within a body for treatment of pulmonary arterial hypertension and/or right heart failure/disfunction. This specification will primarily discuss embodiments of the present invention regarding a shunt connecting a right pulmonary artery (RPA) to a superior vena cava (SVC). However, these embodiments should not be limited to use solely at this location, as use with shunts at other locations is specifically contemplated.

A general example procedure for creating a shunt between a right pulmonary artery and a superior vena cava will be discussed below and further modifications of this procedure and its equipment will then be discussed. In this respect, it is intended that the different embodiments discussed in this specification can be mixed and matched in any combination, particularly with shunt procedure described between a right pulmonary artery and a superior vena cava.

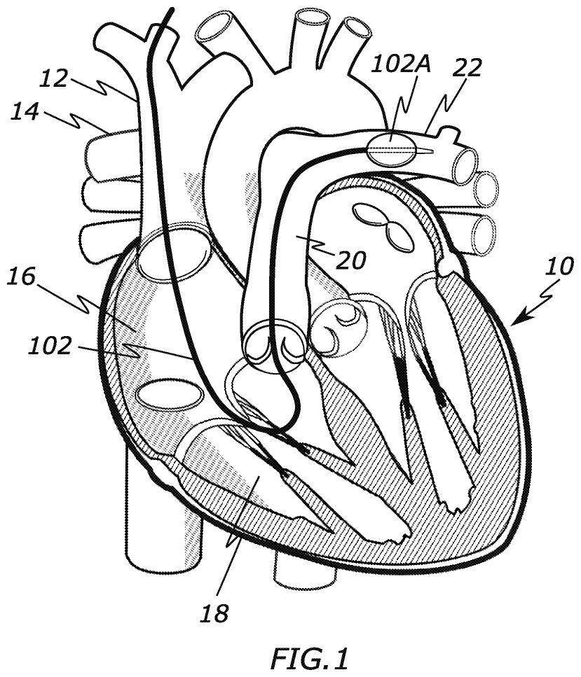

FIGS. 1-12 illustrate various aspects of a method and equipment for creating a shunt 30 between a right pulmonary artery 14 and a superior vena cava 12. While the crossing is performed from the superior vena cava 12 into the right pulmonary artery 14, the opposite may also be performed, crossing from the right pulmonary artery 14 into the superior vena cava 12. The resulting shunt connection may decrease the total pulmonary vascular resistance and the afterload of the right ventricle. The method generally includes the steps of targeting the right pulmonary artery 14, crossing through the superior vena cava 12 to the right pulmonary artery 14 (or vice versa), positioning a shunt support structure 120 between both vessels 12, 14, and removing the delivery system to establish the shunt 30.

Optionally, pre-implant hemodynamic or blood flow-related data may first be acquired from the patient to determine or characterize any abnormalities exist in the heart and lungs. For example, a Swan-Ganz catheterization procedure can be performed, as seen in FIG. 1, to allow pressure measurement in the right atrium, pulmonary artery, and pulmonary capillaries. The pulmonary artery catheter 102 is typically advanced into the right atrium 16 and a balloon tip 102A is inflated, allowing the balloon tip 102A to be carried into the right ventricle 18, into the pulmonary trunk 20, and into the left pulmonary artery 22, which lead to the lungs.

Next, a target and/or grasping device is placed in one of the vessels followed by a piercing device can be placed in the other vessel, allowing one device to pierce both vessels and the other device to grab or engage the piercing device. For example, the target and/or grasping device can be positioned within the right pulmonary artery 14 and the piercing device can be placed in the superior vena cava 12, or vice versa.

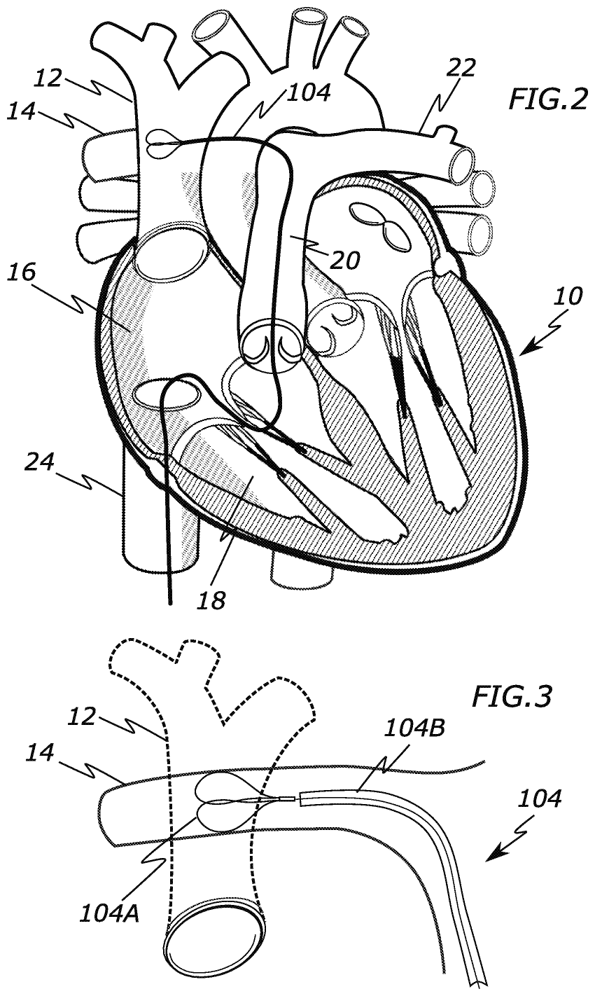

FIGS. 2 and 3 illustrate placing a target and/or grasping device in the right pulmonary artery 14. In this example, the target and/or grasping device is a snare catheter 104 that includes one or more loops 104A that can be retracted into an outer sheath 104B. Additionally, radiopaque markers may be included on the catheter 104 to help “target” the loops 104A of the snare catheter. The loops 104A can be composed of a wire, such as a metal or polymer wire.

The snare catheter 104 can be placed, in one example, through the inferior vena cava 24, into the pulmonary trunk 20, and further into the right pulmonary artery 14. Placement can be achieved with a variety of techniques, including via floating an arrow balloon catheter to the desired location and then advancing the snare catheter 104 within the arrow catheter or other catheter or guidewire advanced to the location of the arrow catheter.

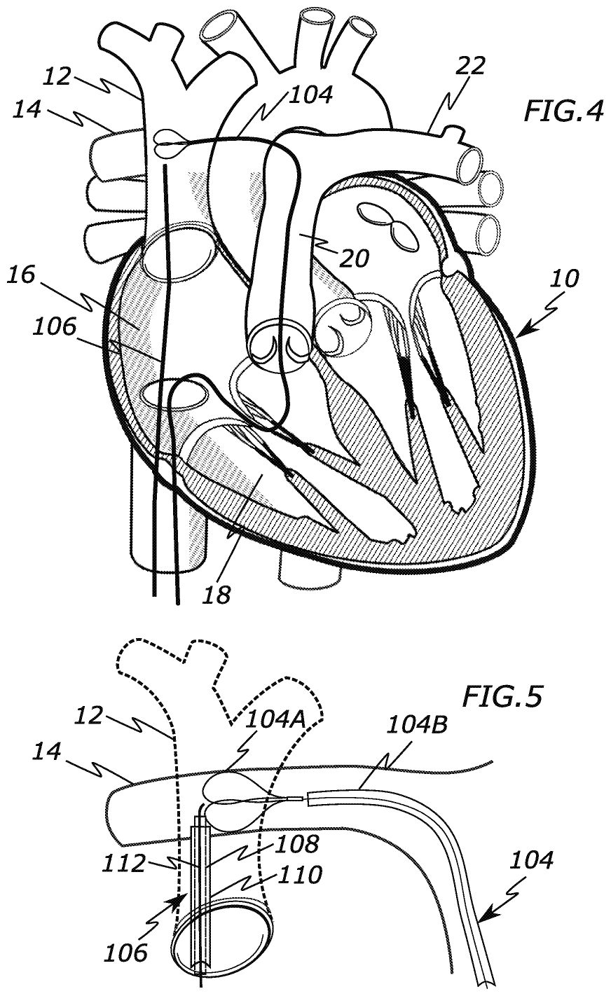

FIGS. 4 and 5 illustrate introducing the puncture system 106 in the superior vena cava 12. In one example, the puncture system 106 may include an outer steerable catheter 110 (e.g., an Agilis catheter), a flexible crossing catheter 108 positioned within the outer steerable catheter 110, and a puncturing guidewire 112 (e.g., an RF guidewire) positioned within the crossing catheter 108. However, other puncturing systems are possible. The puncture system 106 may be introduced by first accessing the femoral vein with a 12Fr catheter sheath. Next, a guidewire (e.g., 0.035″) is advanced to the superior vena cava 12. An outer steerable catheter 110 (Agilis catheter) is tracked over the guidewire into the superior vena cava 12. Finally, the guidewire is exchanged for the crossing catheter 108 and the inner puncturing guidewire 112.

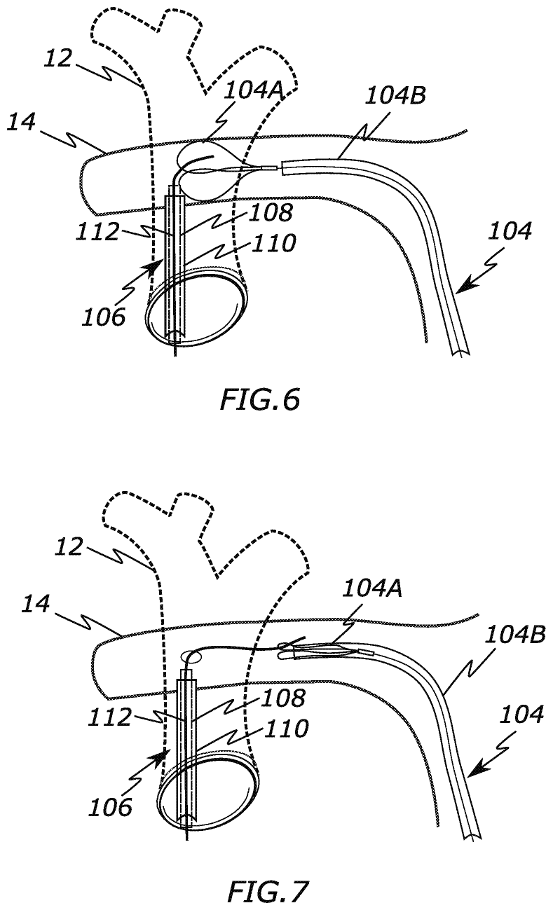

FIGS. 6 and 7 illustrate the process of puncturing the superior vena cava 12 and the right pulmonary artery 14. The tip of the crossing catheter 108 can be angled or directed towards the desired puncture location (e.g., by directing or adjusting the position of the steerable catheter 110) and the tip angle and position can be confirmed via fluoroscopy. Next, the puncturing guidewire 112 is advanced into the target location via its puncturing method (e.g., by activating radiofrequency energy) so that it passes through the wall of the superior vena cava 12, through the wall of the right pulmonary artery 10, and into one of the loops 104A of the snare catheter. The position of the puncturing guidewire 112 within one of the loops 104A can be confirmed via imaging techniques. As seen in FIG. 7, the loops 104 of the snare 104 can be at least partially withdrawn into the outer sheath 104B to grab or capture the puncturing guidewire 112. It also may be necessary to advance the crossing catheter 108 or a different dilator catheter through the punctures to dilate the openings. In that respect, the crossing catheter 108 preferably has a dilating tip.

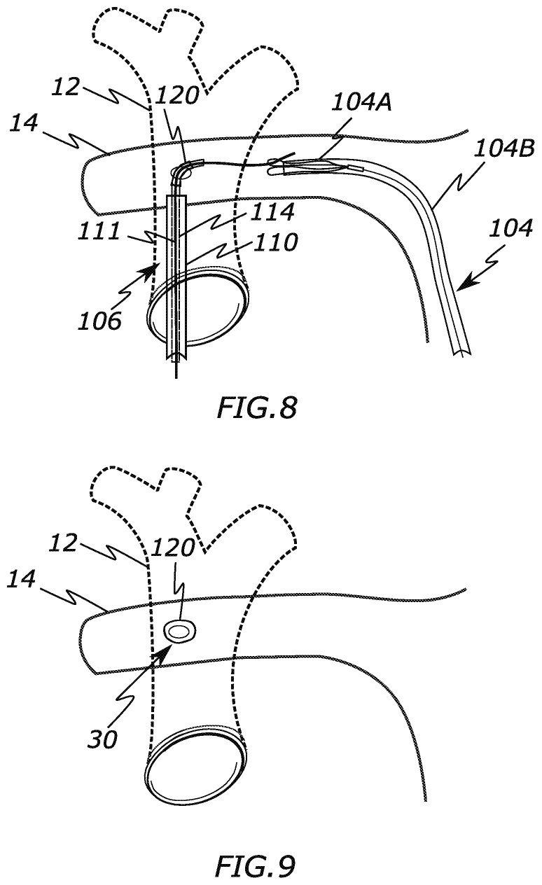

Next, as seen in FIG. 8, a shunt support structure 120 is delivered between the superior vena cava 12 and the right pulmonary artery 14. For example, the puncturing guidewire 112 may be exchanged for a delivery guidewire 111 and the crossing catheter 108 can be removed, allowing a delivery catheter 114 to be advanced over the delivery guidewire 111. The distal end of the delivery catheter 114 is positioned between the superior vena cava 12 and the right pulmonary artery 14. The delivery catheter 114 can have an outer sheath that is withdrawn to expose the shunt support structure 120 and the shunt support structure 120 can be radially expandable by either self-expanding, by a balloon being inflated within the structure 120, or a combination of both methods. The support structure 120 may include a passage therethrough, which creates the shunt passage 30 between both vessels 12 and 14.

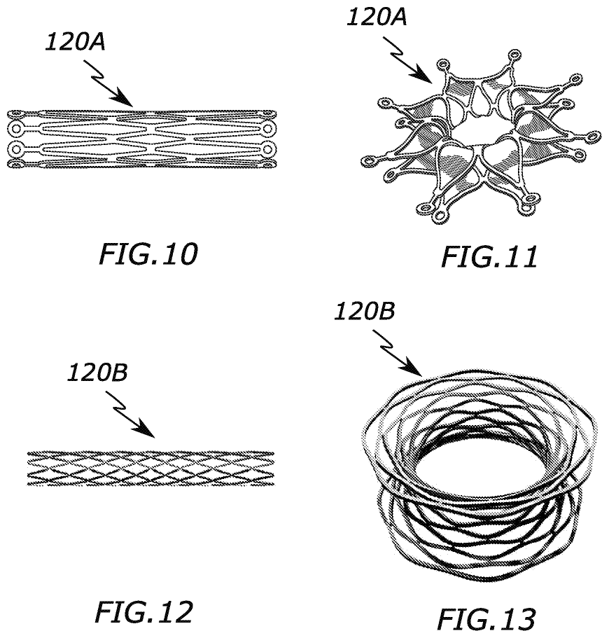

A variety of different shunt support structures 120 are possible. For example, FIGS. 10 and 11 illustrate one embodiment of a structure 120A in a radially compressed and radially expanded configuration, respectively.

Another example shunt support structure 120B can be seen in FIGS. 12 and 13 in a radially compressed and radially expanded configuration, respectively. This structure 120B may expand in a manner similar to a rivet by decreasing in length and radially increasing in size at its proximal and distal ends. Further shunt support structures and details on structures 120A and 120B can be found in U.S. application Ser. Nos. 16/576,704 and 16/785,501, both of which are incorporated herein by reference. Additional shunt methods, techniques, and equipment can also be found in the aforementioned incorporated references.

The following embodiments and methods are discussed in the context of the previously described shunt creation technique and equipment. While only portions of the previously described equipment and procedures are discussed, it should be understood that any or all of the previously described equipment and procedures can be combined with those described below.

In the previous discussion of FIGS. 6-8, the puncturing guidewire 112 is advanced through the superior vena cava 12 and the right pulmonary artery 14, the crossing catheter 108 is advanced through the superior vena cava 12 and into the right pulmonary artery 14, and the delivery catheter 114 is advanced through the punctures of the vessels to deliver the shunt support structure 120. This procedure can be simplified by using a single catheter to both “steer” and dilate/cross the opening created by the puncturing guidewire 112. Such a device may be generally similar to the catheter shown in U.S. Pat. No. 10,076,638, herein incorporated by reference, but may further include a distal tip shape to dilate tissue openings (e.g., a tapered distal tip).

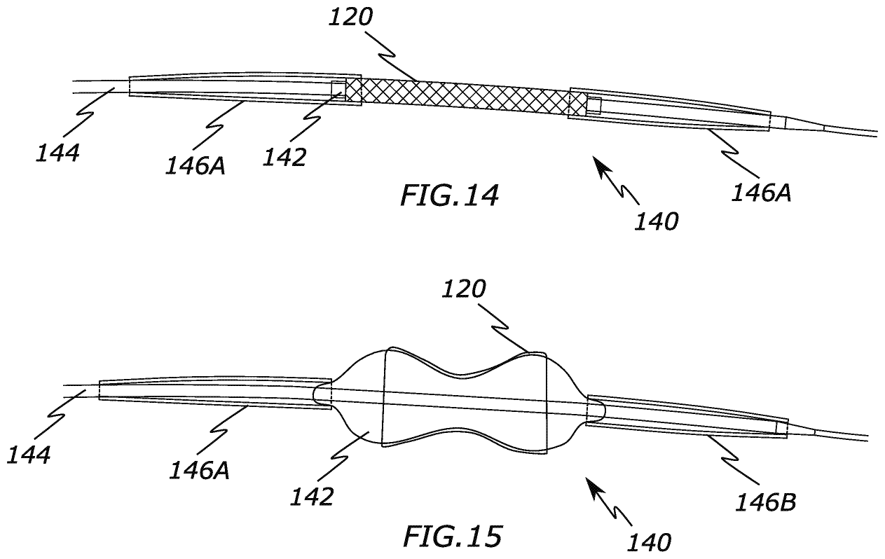

In the previous discussion of FIGS. 6-8, the puncturing guidewire 112 is advanced through the superior vena cava 12 and the right pulmonary artery 14, the crossing catheter 108 is advanced through the superior vena cava 12 and into the right pulmonary artery 14, and the delivery catheter 114 is advanced through the punctures of the vessels to deliver the shunt support structure 120. FIGS. 14 and 15 illustrate one embodiment of a delivery catheter 140 that can cross both vessels 12 and 14 without an overlying sheath disposed completely over the shunt support structure 120 during crossing.

Typically, delivery catheters for stent-like devices include an overlying sheath that completely covers the stent-like device until it is in position for being expanded, at which point the sheath is withdrawn. However, when a delivery device is positioned through the wall of two vessels (e.g., vessels 12 and 14), withdrawing the overlying sheath may pull one or more of the vessel walls, causing the vessels walls to reposition relative to the position of the underlying support structure 120. Hence, minimizing movement against the vessel's walls may help maintain a more consistent position of the vessel walls relative to the shunt support structure 120.

The delivery catheter 140 may include a proximal sleeve 146A and/or a distal sleeve 146B that are each positioned over only the proximal and/or distal ends of the shunt support structure 120 (e.g., 1-5 mm on each end) and radially compressed on a distal end of the catheter 140. A middle portion of the support structure remains uncovered by any protective barrier, such as a sleeve or sheath. This allows most of the shunt support structure 120 to pass through the openings of the vessels “bare”. The sleeves 146A and 146B may be conical in shape, decreasing in diameter away from the structure 120, and may be composed of a relatively soft polymer material.

In one example, the sleeves 146A and 146B are disposed over the elongated body 144 of the catheter 140 in a manner that allows them to slide away from the support structure 120 prior to or during expansion. The one or more of the sleeves 146A and 146B may freely move or slide over the elongated body 144, may be biased to positions covering the support structure 120 (e.g., via a spring or other compressible item positioned within the sleeves 146A and 146B or at either of their free ends), or may have a releasable locking mechanism that releases the sleeves 146A and 146B from a locked position to an unlocked and slidable position (e.g., via a pull wire).

In the example of FIGS. 14 and 15, a balloon 142 is included underneath the support structure 120. The proximal and distal ends of the balloon 142 can be shaped and positioned such that they push the sleeves 146A and 146B away from the support structure 120 when inflated, so as to release the sleeves 146A and 146B from radially retaining the support structure 120. The balloon 142 may also include a tacky or adhesive layer on its outer surface to help further retain the support structure 120 in position on the delivery device 140 during positioning, but while also allowing the support structure 120 to be released during expansion.

Alternately, the proximal sleeve 146A may instead be an outer sheath or catheter with a similar distal position that extends back to a proximal end of the elongated body 144. This outer sheath functions similar to the proximal sleeve 146A except that it is longer. Hence, as the balloon 142 inflates, the outer sheath is proximally pushed back. A bias mechanism, such as a spring, may be connected between the proximal ends of both the outer sheath and the elongated body 144 so as to keep the outer sheath over at least a proximal end of the support structure 120. Additionally, the outer sheath allows the user to manually retract the outer sheath, if necessary, since it extends to the proximal end of the elongated body 144. The distal sleeve 146B may optionally be present in this embodiment.

Alternately, the sleeves 146A and 146B may be configured to remain in place without sliding, but instead at least partially tear as the balloon 142 expands. These sleeves 146A and 146B may be composed of a relatively thin material (e.g., urethane) and may include weakened areas or one or more cuts to promote tearing during expansion.

Alternately, the sleeves 146A and 146B may be configured to remaining in place without sliding or tearing but are instead configured such that the support structure 120 slides out of the sleeves 146A and 146B as the balloon 146A expands. The inner surface of the sleeves 146A and 146B may include a coating to reduce friction and allow slippage. The sleeves 146A and 146B may also be composed of a material that stretches as the balloon 142 expands, allowing the support structure to pull out of the sleeves 146A and 146B as the balloon expands 142.

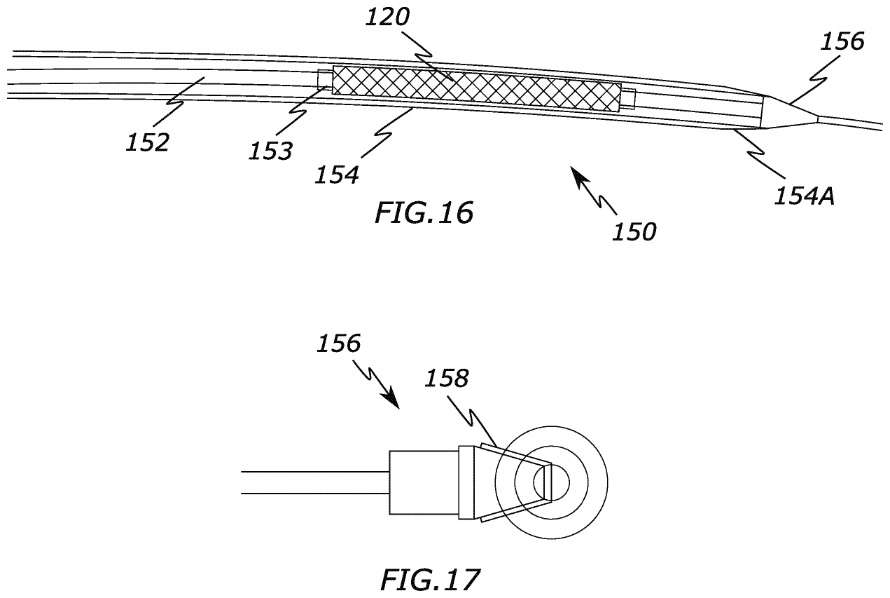

FIGS. 16 and 17 illustrate another embodiment of a delivery catheter 150 that can be used to both pierce the vessels walls of two vessels, such as the superior vena cava 12 and the right pulmonary artery 14, as well as deliver the support structure 120 to both vessels 12 and 14. Hence, instead of the need to use a separate puncturing guidewire or similar device and delivery catheter, only the delivery catheter 150 is needed for the puncture and support structure 120 delivery.

The delivery catheter 150 includes an elongated body 152 with a distal tip 156 configured for piercing vessel walls. In one example, the distal tip 156 includes one or more electrodes 158 that are connected to a power source to supply radiofrequency energy to create an opening in a vessel (e.g., the one or more electrodes 158 are electrically connected to an RF power supply via a proximal end of the catheter).

The delivery catheter 150 can also act as a dilator catheter by having a conical cone that decreases in diameter in the distal direction. Additionally, the delivery catheter 150 may have an outer sheath 154, and therefore to help with dilation, a distal portion 154A of the sheath 154 may be tapered, decreasing in thickness in a distal direction (e.g., along about 2-5 mm in length).

The delivery catheter 150 can also include a support structure 120 that is radially compressed over an inflatable balloon 153. An outer sheath 154 can be withdrawn proximally to expose the support structure 120 and the balloon 153 can be inflated.

In operation, the delivery device 150 is advanced with a vessel, such as the superior vena cava 12 such that its distal tip 156 is angled towards a target or snare catheter in an adjacent vessel, such as a right pulmonary artery 14. The one or more electrodes on the distal tip 156 are activated, e.g., applying radiofrequency energy, to thereby cause an opening in both vessels 12 and 14. The taper of the distal tip 156 and the taper of the distal portion 154A allow the catheter 150 to be pushed through both openings so that it is positioned in both vessels 12 and 14. Next, the outer sheath 154 is proximally withdrawn to expose the support structure 120. Finally, the balloon 153 under the support structure 120 is inflated to expand the support structure (or optionally the support structure is self-expanding). In this manner, the delivery catheter 120 may take the place of several other catheters with dedicated purposes.

As previously discussed in FIGS. 5-8, a puncturing guidewire 112, such as an RF guidewire, can be used to puncture or pass through both the superior vena cava 12 and the right pulmonary artery 14. One danger of using such a RF guidewire is the risk it will contact an unintended area of either of the two vessels 12 and 14 during a procedure, thereby damaging or even creating another opening in one of the vessels 12 and 14. Particularly, there is a danger of extending an RF guidewire longitudinally too far through one or more vessels, such that two openings are created in a vessel.

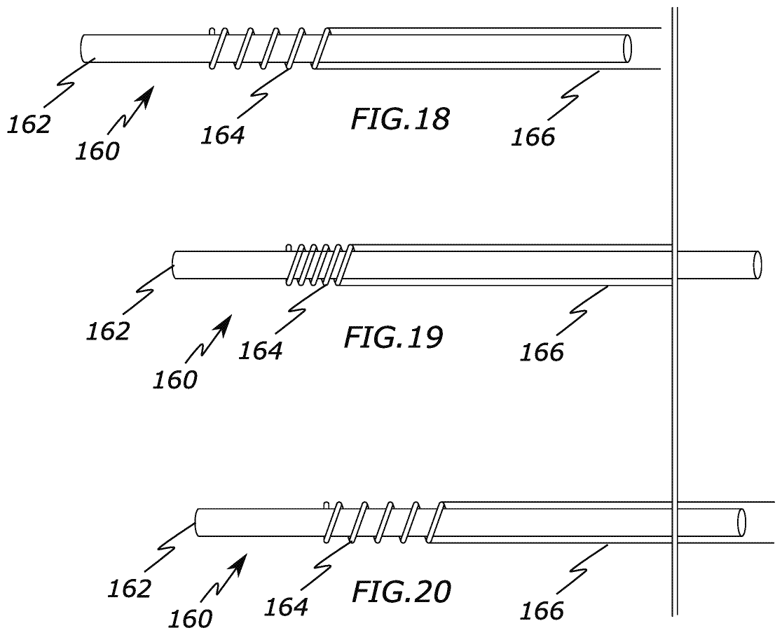

FIGS. 18-20 illustrate one embodiment of an RF puncturing guidewire 160 that includes a protective sheath 166 a distal end of an elongated RF wire body 162 to help protect from unintended lateral contact and unintended longitudinal contact. The sheath 166 is configured to maintain a position such that its distal end is either even with or extends beyond the distal end of the RF wire body 162, as seen in FIG. 18. The distal end of the RF wire body 162 includes one or more RF electrodes that are connected to a power supply, and the sheath 166 thereby prevents contact with tissue in that FIG. 18 position. Preferably, the sheath 166 has a tubular shape for maximum lateral protection, though other configurations are also possible, such as a braided tubular shape.

The sheath 166 is configured to be longitudinally slidable and biased to the FIG. 18 position. For example, a spring 164 or similar compressible element may be fixed to the RF wire body (e.g., at a proximal end of the spring 164) and to the sheath 166 (e.g., at a distal end of the sheath 166), causing the sheath 166 to bias distally. In this respect, the sheath 166 can be configured to longitudinally move only a predetermined distance (e.g., about 1 cm), which may prevent it from passing entirely through the second vessel (e.g., the right pulmonary artery 10).

As seen in FIG. 19, when the distal end of the RF puncturing guidewire 160 is pushed against tissue (e.g., a vessel wall), the sheath 166 moves proximally back only a predetermined distance as the RF wire body 162 pushes against and through a vessel wall (e.g., a stop). The predetermined distance can be configured so as to limit the travel of the RF puncturing guidewire 160, thereby preventing it from advancing too far. As seen in FIG. 20, the sheath can also be pushed through the first vessel wall to cover the distal end of the RF wire body 162 until it is pressed against and through the adjacent vessel.

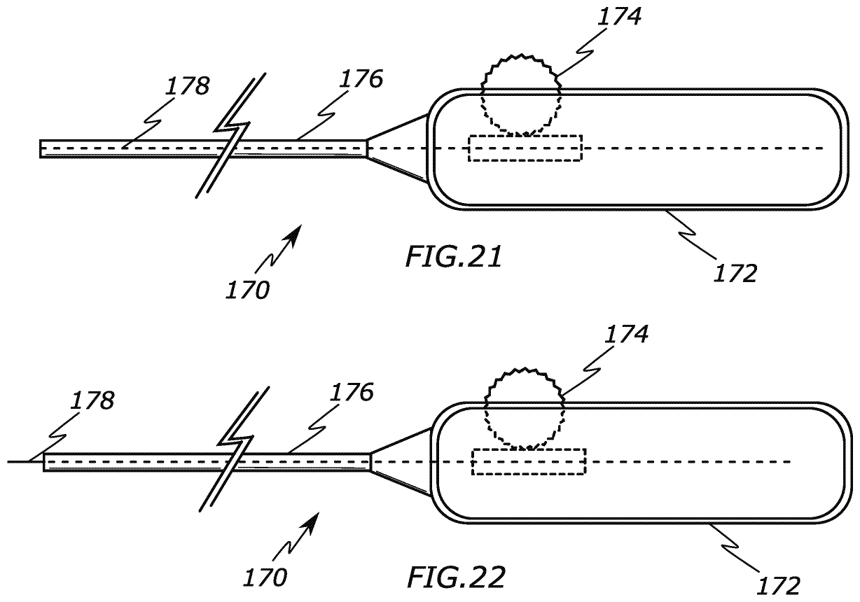

FIGS. 21 and 22 illustrate another embodiment of an RF guidewire assembly 170 that is configured to limit and/or control longitudinal movement of an RF puncturing guidewire to prevent it from distally extending completely through a second vessel (e.g., two walls of a right pulmonary artery 10). Specifically, a handle portion 172 includes a mechanism configured to move the RF puncturing guidewire 178 relative to its outer tubular sheath 176. In one example, the mechanism includes a thumbwheel 174 that engages a toothed track connected to the RF puncturing guidewire 178 such that rotation of the thumbwheel 174 moves the track and the guidewire 178 longitudinally. A limit mechanism or stop member can be positioned within the handle to prevent movement of the guidewire 178 beyond a predetermined distance that would otherwise puncture entirely through a vessel (e.g., 1 cm). Alternate movement mechanisms are possible, such as screw drive mechanisms or thumb sliders. The handle 172 and the guidewire 178 can be connected to an RF power source so as to allow the guidewire 178 to apply RF energy to the patient's tissue.

In practice, the user advances the tubular sheath 178 so that the distal end is in a desired target location. RF energy can be applied to the guidewire 178 so that its distal end can apply radiofrequency energy to tissue. The user can rotate the thumbwheel 174 to cause the RF puncturing guidewire 178 to contact the wall of a first vessel (e.g., superior vena cava 12), pass through its vessel wall, contact a second vessel (e.g., right pulmonary artery 10) and then pass through its wall.

In an alternate embodiment, the handle 172 can move the outer sheath 176 relative to the RF puncturing guidewire 178. This allows the user to advance the entire guidewire assembly 170 to be distally advanced until the distal end of the sheath 178 blocks further advancement.

Alternately or additionally, the guidewire assembly 170 may include a switch or circuit breaker mechanism that interrupts the RF current when the guidewire 178 is extended from the sheath 176 a predetermined distance (e.g., 1 cm). The switch or circuit breaker mechanism may be located within the handle 172 and can be actuated when a portion or feature on or connected to the guidewire 178 distally advances to the predetermined distance. In another embodiment, the switch may be an electrolytic segment of the circuit near or in electrical communication with one of the electrical contacts of the puncturing guidewire 112 or snare, such that as the electrical contacts of the puncturing guidewire 112 contact the snare catheter (e.g., the shield or loops), the electrolytic segment or fuse dissolves, breaking the circuit.

In another embodiment, any of the piercing guidewires discussed in the specification may be connected to an RF energy source with a timer configured to activate for only a length of time sufficient to pierce through one wall of the first vessel (e.g., superior vena cava 12) and/or one wall of the second vessel (e.g., right pulmonary artery 14). For example, the RF energy may be activated for only 0.5 second, 1 second, 1.5 seconds, or two seconds. In this manner, the RF energy can be quickly turned off to prevent unwanted damage (e.g., puncturing entirely through opposite walls of a vessel).

As previously discussed with regard to FIGS. 6-8, a target or snare catheter 104 can be used to capture a puncturing guidewire 112. One challenge with using a snare catheter in this manner is that it may be difficult to maintain the position of its loops 104A so that the puncturing guidewire 112 can be threaded through. Additionally, once through the loops 104A, the puncturing guidewire 112 may be accidentally advanced through the opposite side of the vessel it entered (i.e., entirely through the vessel). The following embodiments address one or more of these challenges.

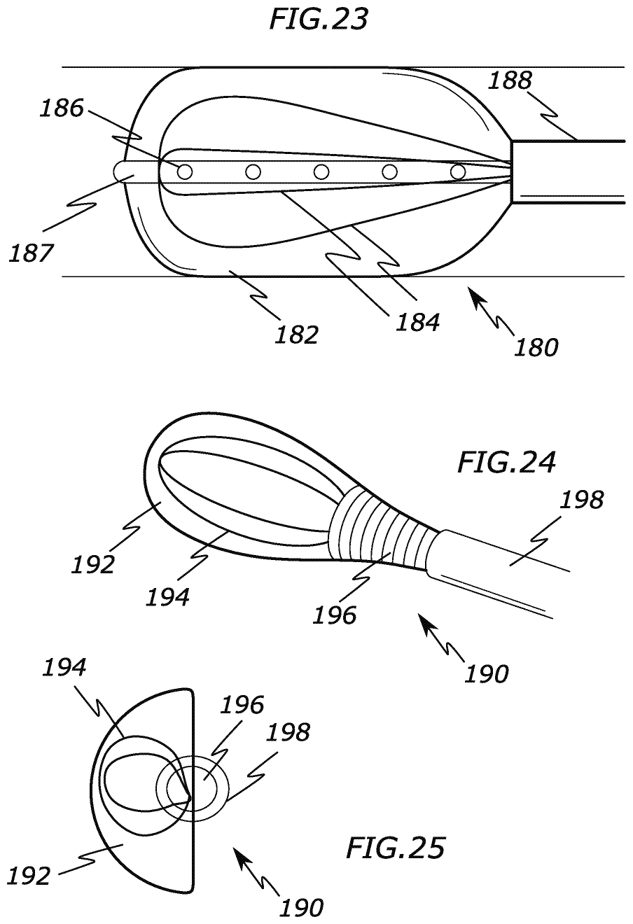

The snare catheter 180 shown in FIG. 23 includes an inflatable balloon 182 that can be inflated to engage the walls of the vessel (e.g., right pulmonary artery 14) so that its distal end can be locked into place within the vessel. The balloon 182 can be located at the distal end of an elongated catheter body 187, which further includes one or more apertures 186 in communication with a fluid passage through the body 188 that allows inflation of the balloon 182. The catheter body 187 can be moved into and out of an elongated tubular sheath 188.

One or more snare loops 184 (e.g., two loops) are positioned at the distal end of the elongated catheter body 187. This can be achieved in several ways. For example, the loops 184 can be fixed to the elongated catheter body 187 and positioned within the balloon 182 such that the balloon 182 inflates around the loops. In another example, the loops 184 may be fixed to the elongated catheter body 187 and positioned outside of the balloon 182 such that the loops remain on an outer surface of the balloon 182 when inflated. In another example, the loops may be positioned outside of and adjacent to the balloon 182 but are connected to a separate elongated body or pusher that allows the loops 184 to move independently of the balloon 182. In another example, the loops 184 can be embedded, adhered to, or bonded to the material of the balloon 182.

In practice, the distal ends of the sheath 188 and catheter body 187 can be positioned at a desired location in a vessel (e.g., right pulmonary artery 14), the balloon 182 can be inflated to engage the walls of the vessel, a puncturing guidewire 112 can be advanced through the loops 184 (and optionally through the balloon 182, and the loops 184 can be at least partially retracted into the sheath 188 to grab the puncturing guidewire 112.

Optionally, the balloon 182 may be composed of a puncture resistant material that resists puncture from the puncturing guidewire 112. For example, only one side may be composed of a puncture resistance material when the loops 184 are located within the balloon 184, allowing the puncturing guidewire 112 to pass through one side of the balloon 184 but not its opposite side. In embodiments with the loops 184 being located outside the balloon 182, the entire balloon may be composed of puncture resistant material. The puncture resistant material may be a hardened polymer or flexible material containing one or more metal strands or panels.

FIGS. 24 and 25 illustrate an alternate embodiment of a snare catheter 190 that includes a rear shield 192 that extends behind a plurality of wire snare loops 194 and blocks a puncturing guidewire 112 from passing entirely through the vessel it is deployed in (e.g., right pulmonary artery 14). Both the snare loops 194 and the shield 192 may be fixed to the end of an inner elongated catheter body 196 which can be extended out of and pulled into an outer tubular sheath 198.

The shield 192 may be composed of a plurality of woven or braided wires, textile, a polymer sheet (e.g., polyurethane), silicone, or similar materials. The shield 192 may also be composed of a shape memory frame (e.g., a Nitinol wire) that allows the shield 192 to expand to its desired shape. The shield 192 may also expand from a radially compressed configuration to an expanded configuration having a variety of different shapes. For example, the shield 192 may expand to an oval, planar shape. In another example, the shield 192 may expand to a curved shape across the axis of the device to conform to the curvature of the vessel it is deployed in, as seen in the end view of FIG. 25.

In one embodiment, the shield 192 can be configured to turn off radiofrequency energy being supplied to a puncturing guidewire 112 that uses RF energy. For example, the shield 192 may be composed of an outer electrically insulated layer and an inner conductive layer so that when the puncturing guidewire 112 punctures through, it creates electrical contact with the conductive layer. The conductive layer and therefore the snare catheter 190 may be connected to an RF power supply that is configured to interrupt the RF power to the puncturing guidewire 112.

The inner catheter 196 may also include a funnel/cone portion at the distal end of its body and proximal of the shield 192 and loops 194 to help radially compress these structures as the inner catheter 196 is pulled proximally back into the outer sheath 198. For example, the funnel may be composed of one or more coiled wires, a braided mesh cone, or a polymer cone.

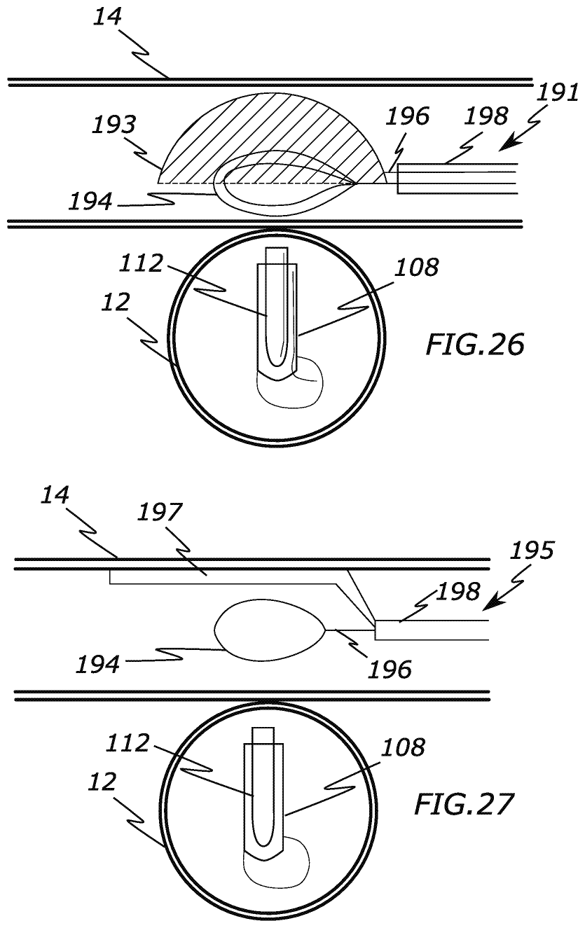

FIG. 26 illustrates an embodiment of a snare catheter 191 that is generally similar to the previously described snare catheter 190 but has a shield 193 forming a circular diameter with a concave interior as opposed to the more oval shape of shield 192. In other words, the shield 193 is hemispherical with an interior space positioned at least partially around loops 194.

FIG. 27 illustrates an embodiment of a snare catheter 195 that is generally similar to the previously described snare catheter 190 but has a generally planar shield 197. The shield 197 can have a variety of different planar shapes, such as a square, rectangle, circle, or oval shape. Optionally, the “plane” of the shield 197 may also have a slight curve and the axial direction of the catheter 195, thereby forming a partial tubular shape.

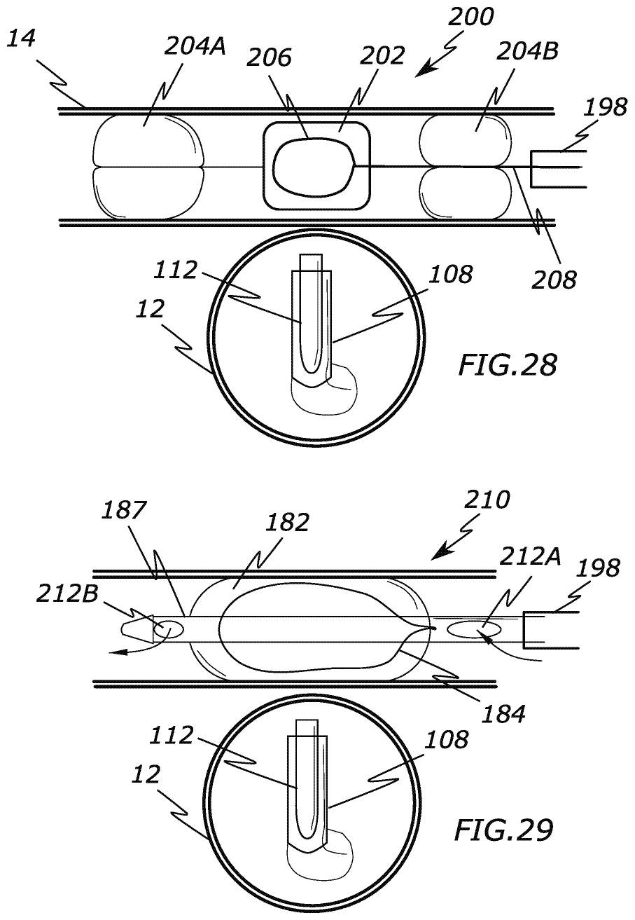

FIG. 28 illustrates an embodiment of a snare catheter 200 that is generally similar to the previously described snare catheter 190 but includes an anchoring mechanism to anchor the shield 202 and snare loop 206 in a desired position within a vessel (e.g., right pulmonary artery 14).

In one example, an elongated inner catheter 208 includes one or more distal balloons 204A and one or more proximal balloons 204B that are spaced on either side of the shield 202 and snare loop 206. The inner catheter 208 includes one or more inflation lumens that are configured to connect to a fluid supply, thereby allowing the balloons to be inflated. Each of the balloons 204A can be a single balloon that entirely expands with in the vessel 14 or can each include a plurality of balloons (e.g., two, three, four, or five balloons). By using a plurality of balloons spaced radially apart, it may be possible to include spaces or perfusion passages across the balloons to allow for blood flow during inflation.

As in any of the previous embodiments, the snare loop 206 can be fixed to the shield 202 or the snare loops 206 can be connected to a separate elongated wire or body that allows it to move independently of the shield 202.

In practice, the distal end of the inner catheter 208 is positioned at a desired shunt creation location, outside of the outer sheath 198. Next, the one or more distal balloons 204A and one or more proximal balloons 204B are inflated to engage the walls of the vessel (e.g., right pulmonary artery 14), distally and proximally of the expanded shield 202 and snare loop 206. The puncturing guidewire 112 is then advanced through another vessel (e.g., superior vena cava 12), into the prior vessel (e.g., right pulmonary artery 14), through the snare loop 206, and is prevented from further advancement by the shield 202. Finally, balloons 204A and 204B are deflated and the inner catheter 208 (or the wire connected to the snare loop 206) is at least partially retracted into the outer sheath 198 to grasp the puncturing guidewire 112.

Any of the embodiments relating to a target or snare catheter may include perfusion features or passages to allow blood to flow around any blockages that are created. While these perfusion features may be particularly desirable for embodiments with balloons (e.g., snare catheter 180 in FIG. 23 or snare catheter 200 in FIG. 28), it may also be desirable in embodiments with a shield as well, since these shields may at least partially block blood flow through the vessel.

As previously discussed for the snare catheter 200, one way to achieve perfusion passages is to provide two or more balloons at a particular location that, when inflated, create gaps or longitudinal passages between themselves. Another technique can be seen in the snare catheter 210 in FIG. 29 which includes a proximal perfusion opening and a distal perfusion opening 212B that both connect to a perfusion passage or channel therebetween in the inner catheter 187. This snare catheter 210 is generally similar to the snare catheter 180 in FIG. 23, but the perfusion channel and openings 212A and 212B can be used on any of the snare catheter embodiments described herein, including those with balloons that also have perfusion passages between themselves.

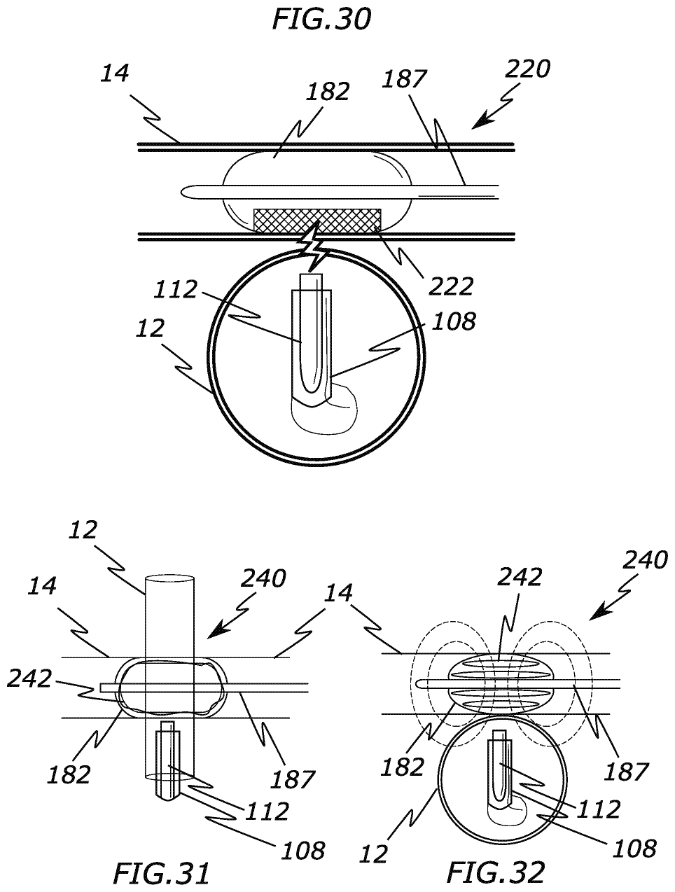

As previously discussed, it can be undesirable for radiofrequency energy from a puncturing guidewire 112 to damage unwanted areas of the patient. FIG. 30 illustrates one embodiment of a snare catheter 220 that helps maintain the RF energy between only the puncturing guidewire 112 and the snare catheter 220 by including one or more RF electrodes 222 in the snare catheter 220. For example, the electrode 222 may be embedded within a balloon 182, a shield, or any component of the snare catheter embodiments of this specification. The one or more electrodes 222 may have an opposite polarity to the electrodes on the distal end of the puncturing guidewire 112 and may also be connected to the RF energy source outside of the patient. Hence, the RF energy takes the path of least resistance to the electrode 222, thereby avoiding other tissue that is not intended to be damaged. The electrodes can be strips of conductive material on or embedded in the balloon 182 (or other component) or can be a plurality of wires arranged in a pattern (e.g., braided).

The snare catheter embodiments of this specification may also include mechanisms for sensing the position of the snare catheter and/or aligning puncturing guidewire 112 with the snare catheter. FIGS. 31 and 32 (side and top views, respectively) illustrate one example of such a snare catheter 240 that creates a magnetic field that can be used for either positioning or self-aligning purposes. In this example, the snare catheter 240 is generally similar to the snare catheter 180 in FIG. 23 except that one or more coils of conductive wire 242 is located within, on, or embedded into the balloon 182. The one or more coils of conductive wire 242 are connected to a power source via the proximal end of the catheter 240, allowing current to selectively pass through the one or more coils 242 and generate a magnetic field.

The magnetic field can be used in two possible ways. First, the puncturing guidewire 112 may include one or more magnetic sensors that can sense the magnetic field, allowing the puncturing guidewire 112 to be better aligned with the snare catheter 240. For example, the one or more sensors may sense the magnitude of the magnetic field on each side of the puncturing guidewire 112 and/or may sense the polarity of the magnetic field, thereby providing additional data to achieve a desired orientation. Second, the puncturing guidewire 112 may have its own magnets or ferrous material that is attracted to the magnetic field generated by the one or more coils of conductive wire 242. This may provide physical force and guidance to better align the puncturing guidewire 112 with the snare catheter 240. Either of these two sensing/aligning features or both of these features can be used.

The coil 242 may also be incorporated into other structures, such as a shield or catheter body. Alternately, either a balloon or shield may include one or more permanent magnets to provide similar functionality. Alternately, ferrous material can be incorporated into the balloon or shield and the puncturing guidewire 112 may include permanent magnets or an electromagnet (e.g., conductive wire coil).

As previously discussed, one challenge of a shunt procedure between vessels, particularly between the superior vena cava 12 and right pulmonary artery 14, is directing the puncturing guidewire 112 through the vessel walls at the desired location and at the desired angle. Further, as the puncturing guidewire 112 is advanced out of the outer steerable catheter 110 (or out of the crossing catheter 108 within the steerable catheter), it may cause the steerable catheter 110 to deflect from the intended position and angle.

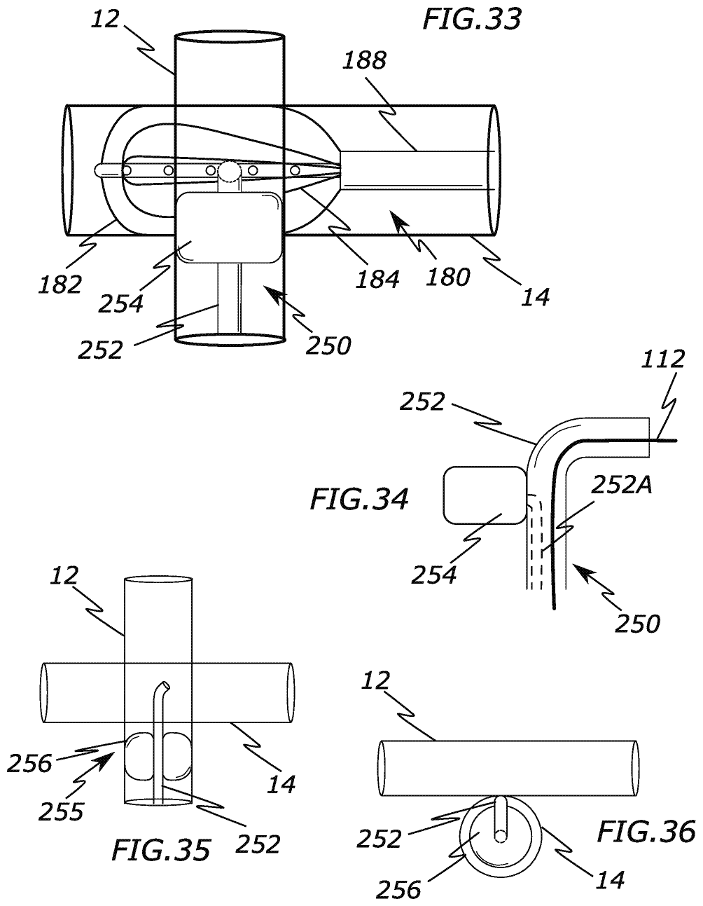

One approach to maintaining the position of the steerable catheter 110 during a procedure is to include an expandable member on a side of the catheter opposite of which it bends forward so as to brace the distal end of the catheter 110 in place. For example, FIGS. 33 and 34 illustrate a steerable catheter 250 having an elongated tubular body with an inflatable balloon 254 that is positioned on an outer surface of the catheter body 252, opposite of the distal opening of the catheter body 252. When the steerable catheter 250 is bent in a first direction, the balloon 254 can be inflated via inflation passage 252A, either prior to or after the bending. The balloon 254 expands in a direction opposite of the bend and braces the back side of the catheter body 252 which allows the puncturing guidewire 112 to be advanced out in a predictable direction and location. The steerable catheter 250 generally comprises an elongated tubular body that includes mechanisms to allow the distal end of the catheter to bend via user controls on a proximal end of the catheter.

FIGS. 35 and 36 illustrate a similar steerable catheter 255 in which one or more balloons 256 inflate on multiple sides of the catheter body 252 to center the catheter body 252 within the vessel 12. Again, this helps provide an anchored position for the steerable catheter 255 that allows for a more predictable location and direction advancement of the puncturing guidewire 112. The one or more balloons 256 can be a single balloon that extends entirely or nearly entirely around the circumference of the catheter body 252, or can be two or more balloons (e.g., 3, 4, or 5 balloons).

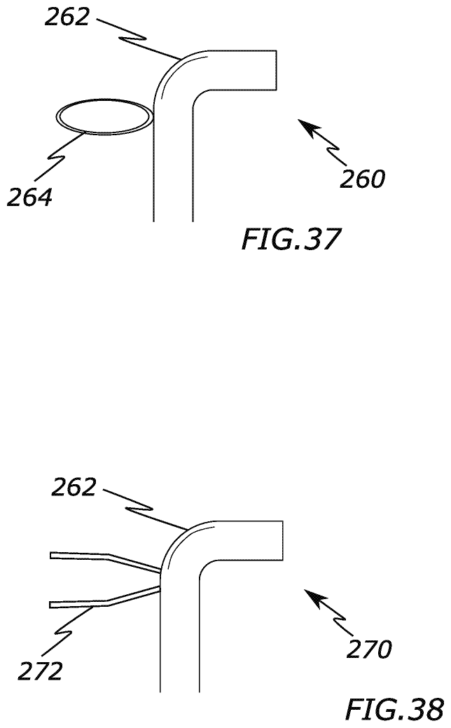

FIG. 37 illustrates another embodiment of a steerable catheter 260 that includes an expandable wire frame or structure 264 that can expand perpendicularly to an axis of the catheter body 262 from a side opposite the bent opening of the body 262. In one example, the expandable wire structure 264 is composed of a shape memory material (e.g., Nitinol) and is shape set to expand to the desired perpendicular position. The wire structure 262 can be a ring shape (e.g., circular, square, etc.). Alternately, the wire structure can be one, two, three, four, or more arms 272, as seen in the steerable catheter 270 in FIG. 38. Each arm can be composed of a shape memory material (e.g., Nitinol) that is biased outwards in a direction generally perpendicular to the body 262. Each arm 272 can be a single wire (e.g., generally straight or bent) or each arm 272 can be a loop of wire (e.g., circular, oval, square, rectangular, etc.).

While the embodiments of the previously discussed FIGS. 33 through 37 are contemplated for use on a steerable catheter through which the puncturing guidewire can be advanced through, other components may also use these features. For example, the snare catheter 104 may also include one or more of these centering or positioning features.

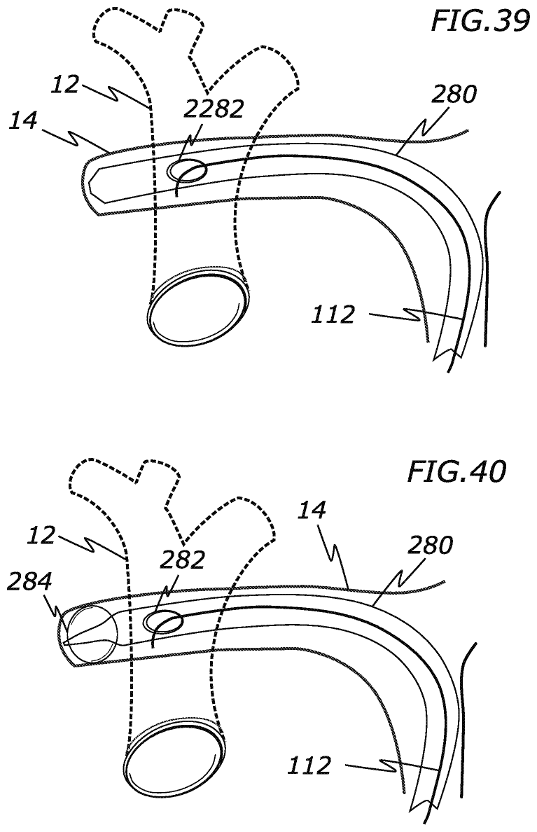

Turning to FIG. 39, a catheter 280 or elongated catheter body having a side aperture 282 can also be used with the shunt creation methods of this specification. The catheter 280 includes at least one lumen within it that is in communication with the aperture 282. The aperture 282 may be located in the sidewall of the catheter, just proximal of the distal end of the catheter 280. For example, the aperture may be about 1-2 cm from the distal end of the catheter 280. The aperture 282 may also have a general diameter of about 0.1-0.5 cm.

In one embodiment, the catheter 280 is configured to form a curve through its distal end to conform to the right pulmonary artery 14 and help brace it during a procedure. In one example, about 5 to 15 cm of the distal end has a curve of about 60-90 degrees relative to the remaining proximal portion of the catheter 280.

In one example use, seen in FIG. 39, the catheter 280 can be advanced into the right pulmonary artery 14 so that the aperture 282 aligns with the superior vena cava 12. Next, a puncturing guidewire 112 is advanced through the lumen of the catheter 280, out the aperture 282, and into the superior vena cava 12.

Optionally, the catheter 280 may include an anchoring device to help brace or maintain its position within the right pulmonary artery 14. One such anchoring device is a balloon 284 that is positioned at the distal end or tip of the catheter 280, as seen in FIG. 40. This balloon 284 is configured to be inflated to a size that engages the vessels walls (e.g., via an inflation lumen in the catheter 280). Alternately or additionally, the catheter 280 may include a balloon, ring, expandable braided mesh, or arms extending from the outer surface of the catheter wall, directly behind the aperture 282.

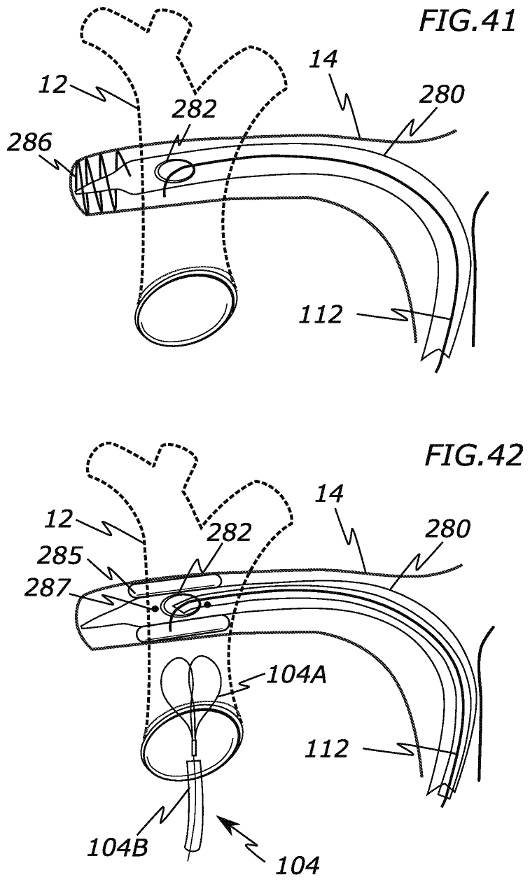

FIG. 41 illustrates another anchoring device comprising wire framework comprising a wire 286 that is attached to and radially expands from a distal end of the catheter 280 to engage the walls of the vessel. The wire may be composed of shape memory material (e.g., Nitinol) and shape set to a desired shape. The shape may include a helical coil, as seen in the figure, a plurality of loops, a plurality of arms, or similar shapes.

FIG. 42 illustrates another anchoring device comprising one or more centering balloons 285 positioned near or adjacent to the aperture 282 so as to position the catheter 280 near a center of the right pulmonary artery 14. Hence, the one or more centering balloons may help both anchor and position the catheter 280 to a position that allows access to the superior vena cava 12. However, the one or more centering balloons 285 may include any of the other anchoring devices previously discussed, as well.

In one example, the one or more balloons 285 is a single “C” shaped balloon that is positioned around the circumference of the catheter 280 at the location of the aperture 285 but leaving the aperture 285 uncovered. In another example, a plurality of cylindrical balloons can be used in a similar position to achieve the “C” shape.

Additionally, radiopaque markers 287 may be included adjacent the aperture 282 in this embodiment or any of the other embodiments. For example, a first marker 287 can be located just distal of the aperture 282 and a second marker 287 can be located just proximal of the aperture 282. Alternately or additionally, markers 287 can be located above or below (i.e., on the same circumference of the catheter 280) of the aperture 282.

As also seen in FIG. 42, a snare 104 (or any of the other snare embodiments of this specification, including those with shields or other safety measures that prevent completely passing through a vessel, such as the embodiment shown in FIG. 26) can be used in the superior vena cava 12 to snare or capture the puncturing guidewire 112. This snare 104 can be used in this manner with any of the previous examples/embodiments.

Again, while the catheter 280 in FIG. 42 is shown in the right pulmonary artery 14, this catheter may also be used in the superior vena cava 12 instead, as any of the embodiments of this specification can be reversed in this manner. In such an arrangement, any of the target/snare catheters described in this specification may be used.

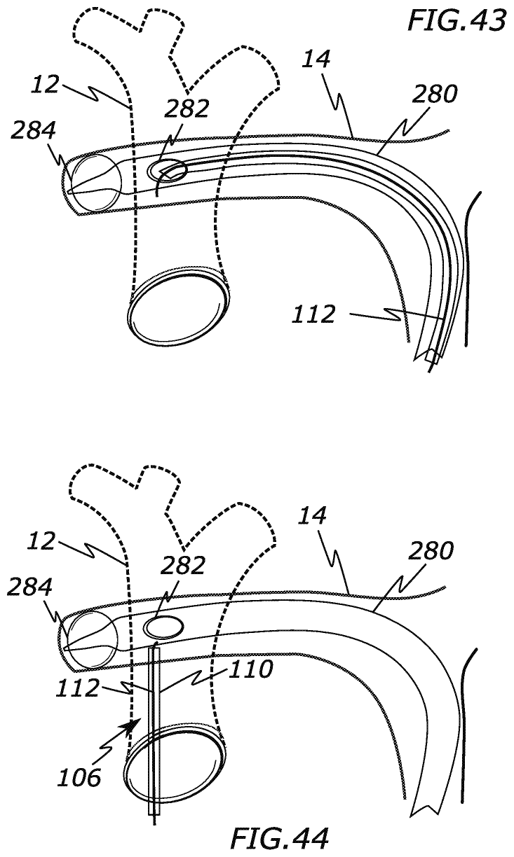

It may be helpful to provide an additional mechanism to help direct the puncturing guidewire 112 out of the aperture 282 in a desired direction. For example, the lumen of the catheter 180 may include a curved or ramped surface near the aperture 282 that is configured to help direct the distal end of the guidewire 112 out of the aperture 282. In another example, the puncturing guidewire 112 may include a balloon, wire loop, or wire arms, extending from one side of its body. In another example, a steerable catheter 110 may be advanced through the lumen of the catheter 280, along with the puncturing guidewire 112, as seen in FIG. 43. In this respect, the distal end of the steerable catheter 110 can be turned or directed so that its distal opening faces or extends out of the aperture 282.

Alternately, the catheter 280 may be used as a target catheter, similar to the previously discussed snare catheter, such that the puncturing guidewire 112 is advanced from the superior vena cava 12 into the right pulmonary artery 14, as seen in FIG. 44.

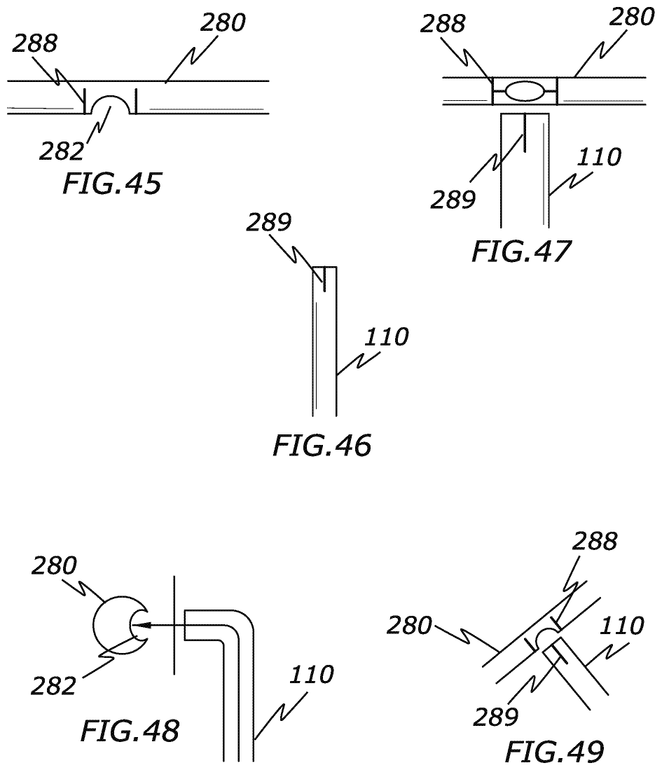

In such an arrangement, it may be desirable to include radiopaque markers on the catheter 280 and on the steerable catheter 110 (or alternately a crossing catheter 108). In one example seen best in FIGS. 45-49, the catheter 280 includes one or more radiopaque marker 288 that are located proximally adjacent and distally adjacent of the aperture 282. For example, the markers 288 may include a first and second line extending perpendicular to the axis of the catheter 280. Additionally or alternately, the markers 288 may include lines parallel to the axis of the catheter 280. The steerable catheter 110 may also include one or more radiopaque markers 289 that allow the user to help line up the distal end of the catheter 110 with the apertures 288 of catheter 280. In one example, the marker 189 is one or more (e.g., 2 or 4) radiopaque lines that are aligned with the axis of the steerable catheter 110. In the case of 2 markers 289, they can be located at about 180 degrees from each other and immediately adjacent to the distal end of the catheter 110. In the case of 4 markers 289, they can be located at about 90 degrees from each other and immediately adjacent to the distal end of the catheter 110.

In practice, the user can view both markers 288 and 289 and then align the markers 189 of the steerable catheter 110 with those markers 288 of the catheter 280. Once aligned (e.g., FIGS. 47-49), the puncturing guidewire 112 can be advanced out of the steerable catheter 110 and into the aperture 282 of catheter 280.

In another embodiment, the catheter 280 may include echogenic markers in similar positions as any of the previously discussed radiopaque markers, either instead of or in addition to the radiopaque markers. The echogenic markers allow a physician to utilize intracardiac echo imaging to monitor and then adjust the position of either of the catheters involved in the procedure.

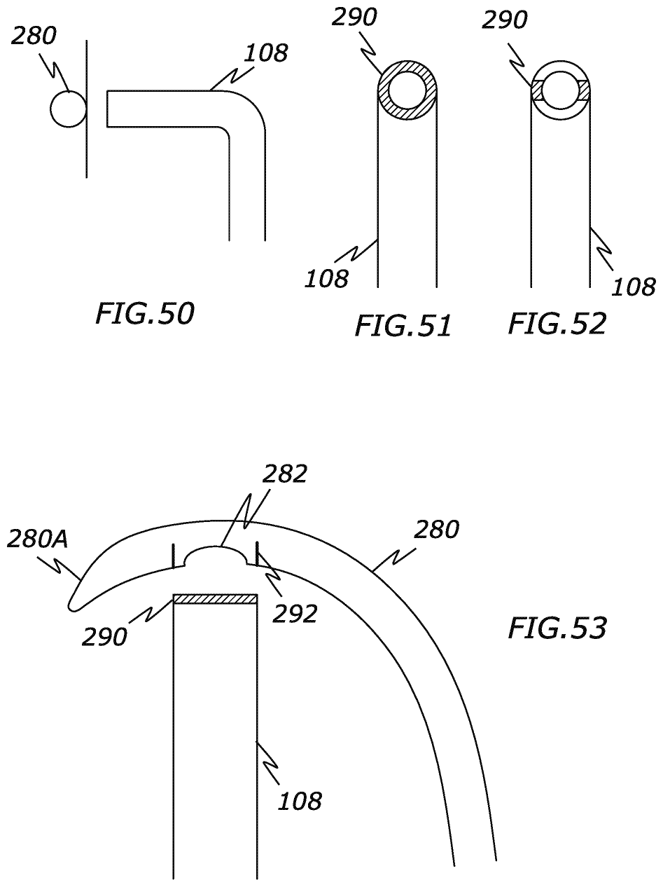

As previously discussed, the catheter 280 can be connected to with a steerable catheter 110 or flexible crossing catheter 108 (or a catheter with both abilities), via a puncturing guidewire 112 passing from either the superior vena cava 14 or right pulmonary artery 14. In either method, a magnetic connection mechanism can be used to help connect to the aperture 282, as seen in FIGS. 50-53. For example, the crossing catheter 108 may include a magnetic ring 290 located at or near the distal edge of the catheter 108. The ring 290 may have magnetic material extending entirely around the distal opening of the catheter 108 as seen in FIG. 51 or the ring 290 may have several discrete areas of magnetic material at locations around the distal opening of the catheter 108, as seen in FIG. 52 (e.g., at least two locations 280 degrees apart from each other).

The catheter 280 may include magnetic material 292 (or ferrous material) near or around the aperture 282. For example, the magnetic material 292 may be two lines or areas proximally and distally adjacent to the aperture 282. Preferably, the magnetic material 292 is spaced apart a similar distance as that of magnetic material 290 on the crossing catheter 290 and configured to attract each other (e.g., opposite polarities), allowing the two areas of magnetic material 290, 292 to align and engage with each other as the tip of the catheter 108 is advanced toward the aperture 282.

The catheter 280 may also include an elongated tip 280A to help position and the catheter 280 in a desired position to achieve a magnetic connection.

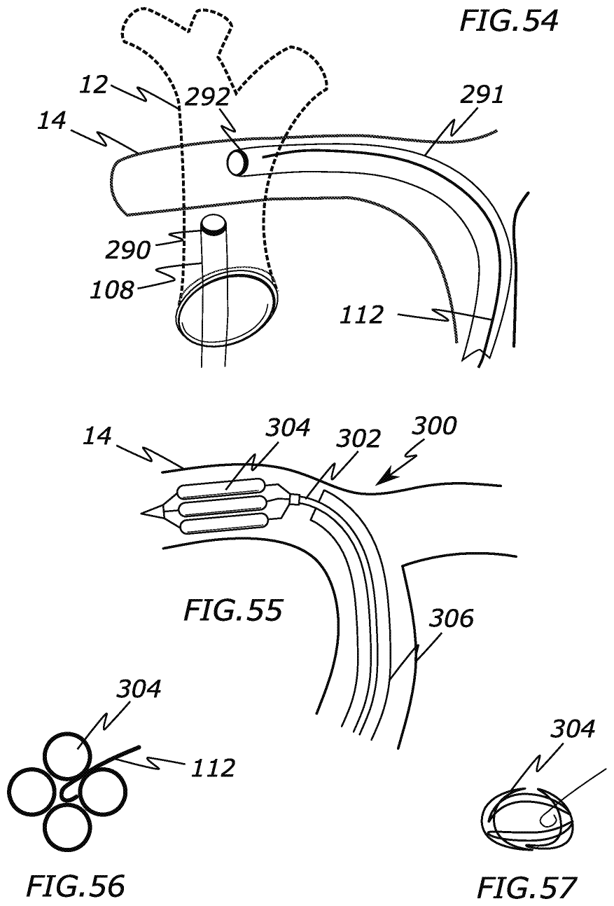

The magnetic material 290, 292 and previous configuration may be included on a variety of different catheter configurations, especially those described in the present specification. For example, two catheters 291, 108 with openings directly on their distal ends can be configured with the magnetic material 290, 192, as seen in FIG. 54. One of more of the catheters 291 and 108 may be steerable (as well as configured for crossing). Hence, the puncturing guidewire 112 can be advanced through either of the catheters 291, 108 and one of the catheters that is configured for crossing/dilating (e.g., crossing catheter 108) can move through the puncture, causing the magnetic material 290 to align with magnetic material 292, connecting the lumens of the two catheters.

FIGS. 55-57 illustrate another embodiment of a target or snare catheter system 300 that captures a distal end of a puncturing guidewire 112 via a plurality of balloons 304. When the puncturing guidewire 112 is positioned between the balloons 304 and the balloons 304 are deflated, they at least partially engage or wrap around the end of the guidewire 112, allowing the elongated catheter body 302 and balloons 304 to be withdrawn into the outer sheath 306, thereby capturing the guidewire 112.

The balloons 304 are positioned at the distal end of an elongated catheter body 302 which includes one or more lumens configured to inflate the balloons 304. The balloons 304 can have a variety of different shapes, including longitudinal cylindrical shapes, as seen in the figures. Preferably, the balloons 204 are positioned adjacent to each other so that after inflation they contact one another but also allows for some space between them so that the guidewire 112 can pass between them and into the space. In one example, the balloons 304 may be supported on a framework (e.g., of tubes or wires) with no central catheter member within the balloon group or alternately, a very small diameter tube/body that allows spacing between it and the balloons 304. The catheter system 300 includes at least two balloons, but three, four, five, six or more balloons 304 are also possible.

FIG. 56 illustrates the guidewire 112 moving into the central space between four inflated balloons after puncturing the walls of the vessels. Once positioned, the balloons 304 are deflated, as seen in FIG. 57, which cause the balloon material to partially wrap around the guidewire 112. The elongated catheter body 302 and balloons 304, along with the captured guidewire 112 are retracted into the outer sheath 306 to further lock the position of the guidewire 112.

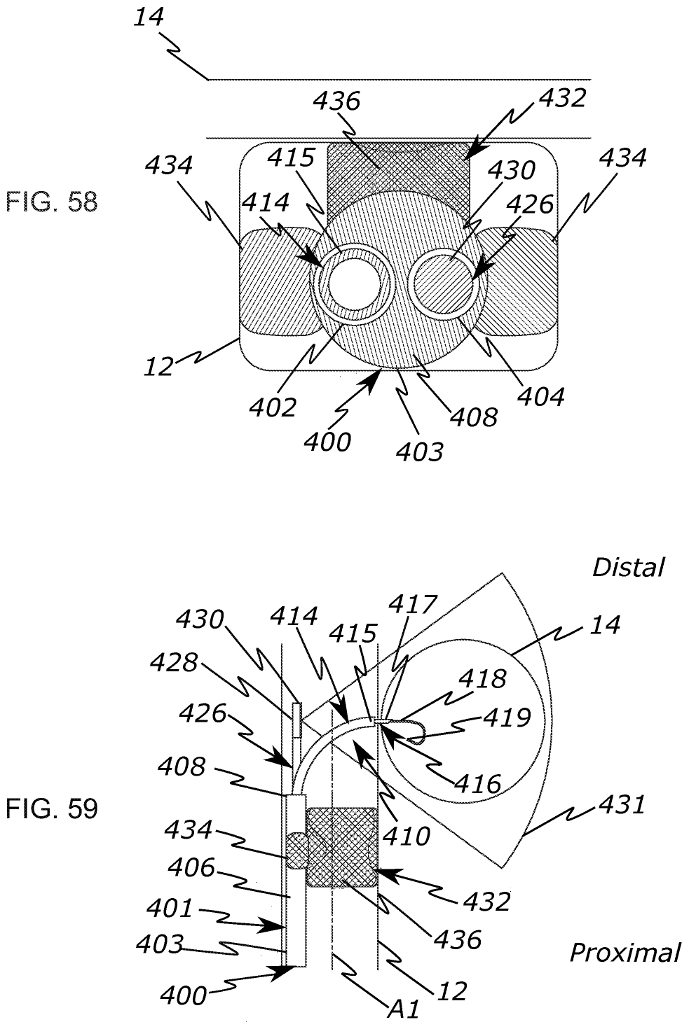

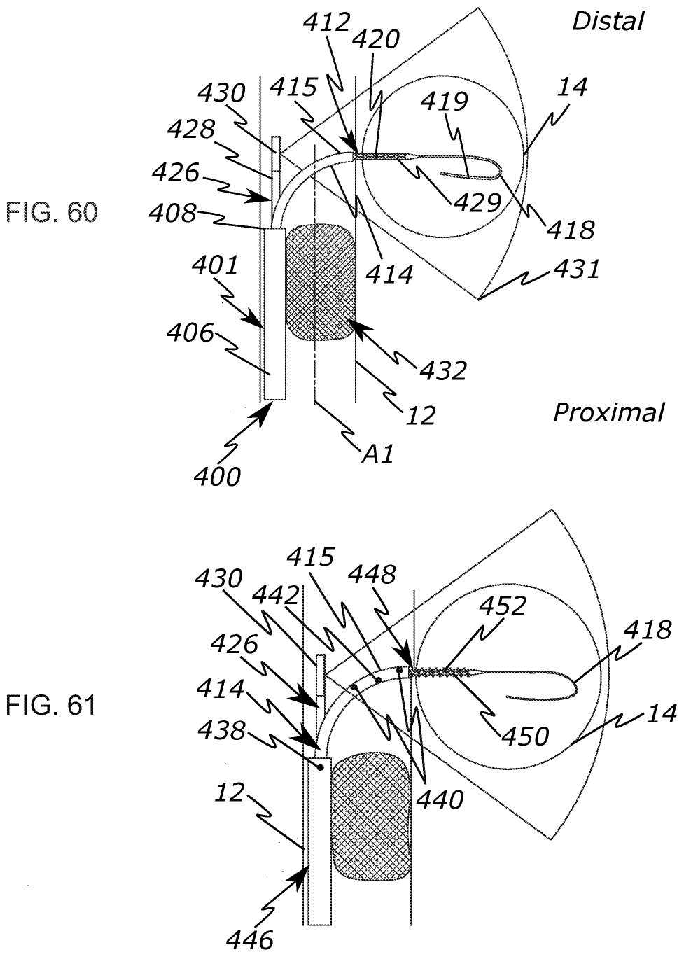

FIG. 58 illustrates a top cross-section of a catheter system 400 including a catheter sheath 401 in the superior vena cava 12. FIGS. 59-60 illustrate side views of the catheter system 400 of FIG. 58 in the superior vena cava 12. FIGS. 58-60 are discussed below concurrently. Also shown in FIGS. 59-60 are orientation indicators Proximal and Distal, relating to relative positions along the catheter sheath 401, and a central axis A1 defined by the superior vena cava 12. In some examples, the catheter sheath 401 may be a dual-lumen guide sheath or tube defining a first lumen 402 (FIG. 58) and a second lumen 404 (FIG. 58). The first lumen 402 and the second lumen 404 may extend axially within the catheter sheath 401, such as through a proximal portion, a distal portion 406 (FIGS. 59-60), and a distal end 408 thereof.

In some examples, the first lumen 402 and the second lumen 404 may each extend parallel to, and laterally offset from, each other within the catheter sheath 401. In other examples, the catheter sheath 401 may include additional lumens extending axially therethrough, such as, but not limited to, three or four individual lumens for receiving other catheters or devices, such as a separate balloon catheter for temporarily sealing or blocking blood flow there past. The first lumen 402 and the second lumen 404 may be sized and shaped similarly or differently relative to each other, such as depending upon the type or style of catheter, or any other elongated devices, that the first lumen 402 and the second lumen 404 may be configured to receive.

In some examples, the first lumen 402 may be adapted (e.g., sized and shaped) to receive a puncturing system 410 (FIG. 59) and/or a deployment system 412 (FIG. 60). While some specific examples are described below, it is to be appreciated that the puncturing system 410 may be representative of, or may include, any of the puncturing systems, puncturing guidewires, or crossing catheters or other devices discussed in this disclosure. Similarly, the deployment system 412 may be representative of any deployment or delivery catheter capable of deploying a shunt support structure or engaging with a snare catheter, or any other targeting device, discussed in this disclosure.

In one example, the puncturing system 410 may include an outer steerable catheter 414, a crossing catheter 416 translatable within the outer steerable catheter 414, and a guidewire 418 including a distal tip 419. In some examples, the outer steerable catheter 414 may be an Agilis catheter (such as corresponding to the outer steerable catheter 110), the guidewire 418 may be a puncturing guidewire (such as corresponding to the puncturing guidewire 112) including a distal tip 419 (e.g., an RF tip), and the crossing catheter 416 may be a flexible crossing or dilating catheter translatable within the outer steerable catheter 414 (such as corresponding to the flexible crossing catheter 108), such as after vessel puncturing via the guidewire 418 has occurred. In another example, the crossing catheter 416 may be translatable within the outer steerable catheter 414 and may include a puncturing tip 417 through which the guidewire 418 may be advanced. In such an example, the guidewire 418 may be a non-puncturing or ordinary guidewire and may be advanced through the puncturing tip 417 after vessel puncturing has occurred.

In some examples, the outer steerable catheter 414 may be at least partially manufactured using laser cutting, such as integrally laser cut with a pull wire thereof; and may be steerable via actuation of the pull wire and/or various user controls on a proximal end thereof. In another example, the puncturing system 410 may not include the crossing catheter 416, and the outer steerable catheter 414 may a steerable catheter adapted to receive only the guidewire 418 for vessel puncturing operations (via distal tip 419). In a further example, the puncturing system 410 may include only the crossing catheter 416 and the guidewire 418, and in such an example, the crossing catheter 416 may be a steerable or a deflectable catheter. In still further examples, the outer steerable catheter 414 and/or the crossing catheter 416 may also include any feature, or aspect, of any steerable, deflectable, targeting, or crossing catheter previously described above or described with reference to additional examples below. Moreover, the guidewire 418, including the distal tip 419 thereof, may include any feature or aspect of any puncturing or non-puncturing guidewire previously described in the present disclosure above.

In various examples, the distal tip 419 may be a solid non-RF or RF tip, the puncturing tip 417 may be a hollow RF or non-RF tip, or the distal tip 419 may be a retractable spring-loaded tip, such as or similar to the puncturing guidewire 160. In one example, the distal tip 419 of the guidewire 418 may be shape set, or may otherwise have a shape memory, configured to cause the distal tip 419 to curve or curl significantly, such as shown in FIGS. 58-60, such as upon exiting the puncturing tip 417 of the crossing catheter 416, or in other examples, the outer steerable catheter 414 to help prevent overpenetration. Further, in various examples, any of the devices or components of the catheter system 400 may be pre-shaped, or may otherwise have a shape memory, to cause such devices or components to automatically return to a pre-set shape, such as curved or angled relative to a central axis thereof, to help the device or component to conform to, or better align with, a known curvature or shape of a vessel in which it is to be used.

In some examples, the deployment system 412 may be representative of, but not limited to, any of the delivery catheter 114, the delivery catheter 120, or the delivery catheter 140 previously discussed above. For example, the deployment system 412 may be a delivery catheter translatable within the outer steerable catheter 414 and including a shunt support structure 420 (such as corresponding to shunt support structure 120A or 120B in FIGS. 10-13) located about a distal portion 429 thereof. In another example, the deployment system 412 may be an RF enabled puncturing delivery catheter, such as or similar to the delivery catheter 150, such as preloaded into the outer steerable catheter 414, as, in such an example, the catheter system 400 may not include puncturing guidewire or puncturing crossing catheter. In some examples, the deployment system 412 may or may not include a sheath entirely covering the shunt support structure 420, or one or more proximal or distal cones that covering only proximal and/or distal ends of the shunt support structure 420, or other devices for helping to retain the shunt support structure 420 in position on the deployment system 412 during advancement of the deployment system 412.

The shunt support structure 420, such as previously described or that referenced with respect to any of FIGS. 9-17, may be radially expandable when at a desired deployment location, either by self-expanding via shape memory or via expansion of an expandable element therein, such as an expandable balloon located within the shunt support structure 420, or a combination of both expanding methods. In some examples, such an expandable balloon may be, but not limited to, the balloon 142 of the delivery catheter 140 or the balloon 153 of the delivery catheter 150, may also include tacky engagement regions or adhesive areas along an outer surface thereof, such as to help retain the shunt support structure 420 in a position along the deployment system 412, while also allowing the shunt support structure 420 to be released therefrom upon radial expansion thereof.

In some examples, such engagement regions or areas may include, or may be comprised of, a different (e.g., softer) durometer relative to other areas. In further examples, such engagement regions or areas may further define friction generating elements, projections, or patterns. In some examples, the deployment system 412 may be adapted to slide over the guidewire 418 after the outer steerable catheter 414 and/or the crossing catheter 416 of the puncturing system 410 are proximally withdrawn, through the catheter sheath 401, over the guidewire 418. In alternative examples, the deployment system 412 may include a separate guidewire adapted for insertion through the outer steerable catheter 414, after the guidewire 418 and/or the crossing catheter 416 have been withdrawn from the catheter sheath 401.

The second lumen 404 of the catheter sheath 401 may be adapted (e.g., sized and shaped) to receive an imaging catheter 426. The imaging catheter 426 may be representative of a variety of different types or styles of imaging catheters, or other imaging device adapted for performing navigational or other imaging operations with vasculature or other anatomy of a patient. In some examples, the imaging catheter 426 may be a commercially available imaging catheter, such as, but not limited to, the a VeriSight ICE catheter available from Koninklijke Philips N.V. In other examples, the imaging catheter 426 may be a proprietary, or a custom-built, imaging catheter or other device adapted for use with the catheter sheath 401. In one such example, the imaging catheter 426 may be a separate catheter that is freely translatable within, and movable distally beyond, the second lumen 404 of the catheter sheath 401. In another example, the imaging catheter 426 may be a device, not limited to a catheter, that is preloaded into, or is otherwise integrated at, the distal end 408 or the distal portion 406 of the catheter sheath 401. In such an example, the imaging catheter may or may not be distally advanceable beyond the distal end 408.

The imaging catheter 426 may include a distal region 428 (FIGS. 59-60) including an imaging head 430. In some examples, the imaging head 430 may be a distal-most end, segment, or region of the imaging catheter 426. In some examples, such as in any of FIGS. 58-67, the imaging head 430 may be configured to perform imaging operations when the imaging head 430 is located parallel to the central axis A1. The imaging head 430 may include various operative components or devices configured to enable the imaging catheter 426 to perform imaging techniques including, but not limited to, phased array, or radial or rotational, Intracardiac Echocardiography (hereinafter “ICE”) imaging, or alternatively, Intravascular Ultrasound (“UVUS”) imaging.

The imaging head 430 may carry out its imaging operations (e.g., provide visual imaging data to a screen) within a field of view 431, the size and shape of which may depend upon a type of imaging that the imaging catheter 426 is configured to perform. In one example, the imaging head 430 may be configured to perform phased array ICE imaging, and the imaging head 430 have a field of view 431 measuring about 90 degrees at the imaging head 430, such as shown in FIGS. 59-60, and extending normally to the central axis A1. In such an example, this may enable a physician to visualize a target vessel within the field of view 431, such as the right pulmonary artery 14, from an anterior-posterior direction or view. In another example, the imaging head 430 may be configured to perform radial or rotational ICE imaging, and the field of view 431 may extend 360 degrees around the imaging head 430. In such an example, a field of view may be a 360-degree radial view located or extending normally to field of view 431 as shown in FIG. 59-60, and a physician may visualize a target vessel, such as the right pulmonary artery 14, from an inferior-superior direction or view.

The catheter sheath 401 may, in some examples, include an anchoring system 432. The anchoring system 432 may be representative of any of the centering, anchoring, bracing, or otherwise expandable components of any puncturing, targeting, crossing, or delivery catheter previously discussed above. For example, the anchoring system 432 may generally include a plurality of balloons or self-expandable structures configured to extend radially outwardly from the outer surface 403 to engage a vessel wall, such as, but not limited to, one or more balloons, arms, laser cut or welded expandable wireframes or wire frameworks, rings, braided meshes or braided segments, or loops that may self-expand, may be expanded via of inflation of a balloon, to limit relative axial and/or lateral movement of the catheter sheath 401 within vasculature of a patient by contacting and engaging a wall of a blood vessel.

In one example, such as shown in FIGS. 58-60, the anchoring system 432 may include one or more balloons 434 and one or more secondary elements 436 each located near the distal end 408 of the catheter sheath 401. In some examples, such as shown in FIGS. 58-60, the one or more balloons 434 may be centering balloons located 180 degrees offset from each other and adapted for helping to position the catheter sheath 401 in a particular location within a vessel, such as centrally aligned with, or axially offset from, the central axis A1. The one or more balloons 434 may include, for example, but not limited to, one, two, three, four, five, six, or other numbers of balloons spaced radially apart from one another about an outer surface 403 of the catheter sheath 401. In another example, the one or more balloons 434 may be a single balloon that may engage an entire circumference of a wall of the superior vena cava 12, or any other blood vessel in which the catheter system 400 may be located. In such an example, the balloon may include perfusion passages adapted to enable blood to continue flowing there past. In any example of the catheter system 400 include inflatable anchoring balloons about the outer surface 40, the catheter sheath 401 may also include one or more inflation lumens extending therein for communicatively connecting the at least one balloon to a fluid supply to thereby enable the at least one balloon to be inflated.

The one or more secondary elements 436 may be, such as shown in FIGS. 58-60, at least one expandable wire framework located 90 degrees offset from the one or more balloons 434. Such an expandable wire framework may form a stent-like or mesh structure (e.g., a plurality of struts interconnected together to form a plurality of diamond shapes or open cells therebetween). In another example, the secondary element 286 may be representative of one or more expandable wires or coils, such as or similar to the wire 286 (FIG. 41). In further examples, the one or more secondary elements 436 may include one or more of the bracing or anchoring features shown in FIGS. 33-37, such as a ring shape structure (e.g., circular, square, etc.) similar to the wire structure 262, or a structure including one, two, three, four, or more arms 272. In still further examples, the anchoring system 432 may include any combination of anchoring balloons or secondary elements. Moreover, in any of the above examples, it may be desirable to radially space the various vessel engaging elements of the anchoring system 432 to form perfusion passages therebetween, such as enable blood flow there past when the anchoring system 432 is expanded.

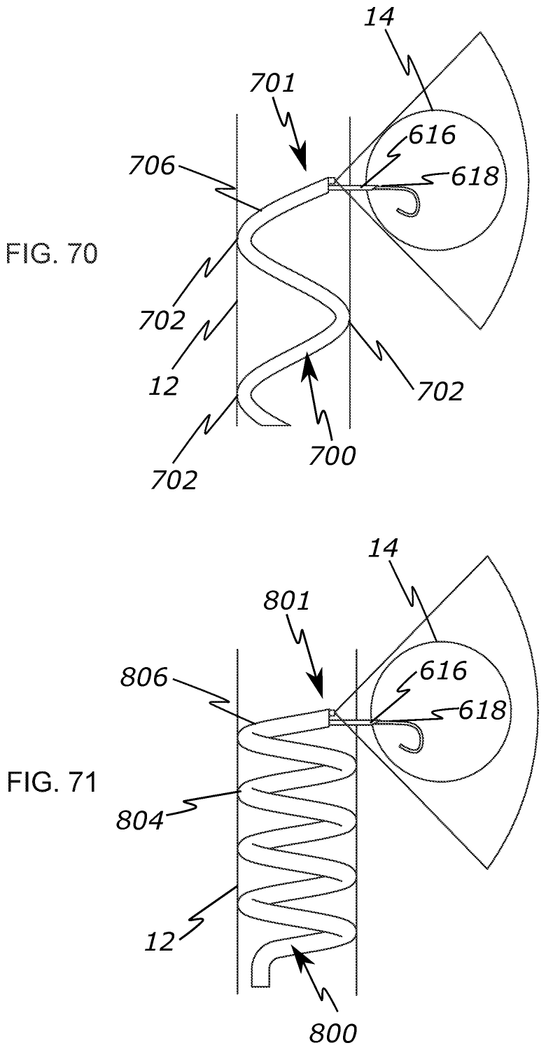

In still further examples, the catheter sheath 401 may be pre-shaped otherwise to directly contact and engage, via the outer surface 403 thereof, a wall of the superior vena cava 12, or any other vessel in which the catheter system 400 may be positioned to help brace or prevent movement of the distal end 408 of the catheter sheath 401 during advancement or use of the puncturing system 410, or during advancement of the deployment system 412. In one such example, the distal portion 406 of the catheter sheath 401 may form an alternating S-shape, such as similarly to the catheter body 701 of the catheter assembly 700 shown in FIG. 70. In another such example, the distal portion 406 of the catheter sheath 401 may form a helical shape, such as similarly to the catheter body 801 of the catheter assembly 800 shown in FIG. 71. In the above examples, it is to be appreciated that, in contrast to the catheter body 701 (FIG. 70) and the catheter body 801 (FIG. 71), a significant length of the distal portion 406 adjacent the distal end 408 and housing the imaging head 430 may remain parallel to the central axis A1 to enable the catheter system 400 to function as described above.

FIGS. 59 and 60 at least partially illustrate an example method of creating a shunt between the superior vena cava 12 and the right pulmonary artery 14 using the catheter system 400. First, the catheter sheath 401 of the catheter system 400 may be inserted into a patient via an access point, such as in a femoral vein in a leg of a patient, such as described with respect to FIGS. 4-5, or alternatively, in a jugular vein in a neck of the patient, such as to access the superior vena cava 12 from a downward direction opposite the upward direction shown in FIGS. 4-5. The catheter sheath 401 may then be advanced distally into the patient, such as until the distal end 408 of the catheter sheath 401 enters a desired vessel, such as the superior vena cava 12.

The position of the catheter system 400 may then be tuned or adjusted, using the images generated by the imaging catheter 426, to position the distal end 408 in a location proximal to a target vessel, such as adjacently to the right pulmonary artery 14. For example, the imaging catheter 426 may first be inserted into, and advanced distally through, the second lumen 404 of the catheter sheath 401, such as until the imaging head 430 enters the superior vena cava 12 through the distal end 408. Next, using the imaging catheter 426, a physician may determine (e.g., visualize on a screen within the field of view 431) a relative position of imaging head 430 relative to the pulmonary artery 14 or other anatomical features located externally nearby the superior vena cava 12.

In response, the physician may advance, or retract, the catheter sheath 401 within the superior vena cava 12, such as until the physician is satisfied that the imaging head 430 is positioned at a height about equal to a center of the right pulmonary artery 14. In one example, such as shown in FIGS. 59-60, this may be when the right pulmonary artery 14 is centered within the field of view 431 of the imaging head 430, such as in a phased array configuration of the imaging head 430. In other examples, this may be when the right pulmonary artery 14 is visible to the physician, such as in a radial or rotational configuration of the imaging head 430.

The catheter system 400 may then optionally be anchored within the superior vena cava 12, such as via the inflation of the one or more balloons 434 or the expansion of the one or more secondary elements 236 to help prevent relative movement between the catheter sheath 401 and the superior vena cava 12, and thereby, provide for increased stability and predictability during the subsequent advancement of the puncturing system 410 and/or the deployment system 412. Next, the puncturing system 410 may be advanced distally beyond the distal end 408 of the catheter sheath 401 into the superior vena cava 12. In some examples, the distal portion 415 of the outer steerable catheter 414 may first be directed towards a desired puncture location into the right pulmonary artery 14, such as by selectively curving, deflecting, or otherwise angling (e.g., via actuation of a wire or control mechanism, or inflation of a balloon) the distal portion 415 toward a wall of the superior vena cava 12 adjacent the right pulmonary artery 14. Moreover, during such advancement or deflection, the distal portion 415 may be continuously viewed (e.g., visualized on screen) to enable a physician to selectively direct or position the outer steerable catheter 414 at a desired puncture location.

In some examples, when fully deflected or curved, the distal portion 415, or a distal tip or portion of an alternative steerable device, may be orthogonally deflected or positioned relative to the central axis A1 and the distal end 408 of the catheter sheath 401. This may help to ensure that, when the crossing catheter 416 or the guidewire 418 are advanced through the outer steerable catheter 414, the puncturing tip 417, or the distal tip 419 are aligned with a center of the right pulmonary artery 14 as visualized through the imaging head 430. In some examples, before proceeding to puncturing operations, a physician may confirm a final position of the outer steerable catheter 414 relative to a vessel wall, such as by confirming the amount of deflection or curvature of the distal portion 415, as well as the direction of the deflection or curvature of the distal portion 415, by viewing the outer steerable catheter 414 via the imaging head 430, and/or by viewing the outer steerable catheter 414 through fluoroscopic imaging, such as performed with a C-arm.

Subsequently, a puncturing device, such as, in some examples, the puncturing tip 417 of the crossing catheter 416 or alternatively the distal tip 419 of the guidewire 418 or may be advanced through the outer steerable catheter 414, into the wall of the superior vena cava 12, through and an adjacent wall of the right pulmonary artery 14 into the right pulmonary artery 14. In some examples, the position of the distal tip 419 or the puncturing tip 417 may also be confirmed at a location outside the distal portion 415, before being advanced through the wall of the superior vena cava 12, may further be confirmed through fluoroscopic imaging, such as performed externally with a C-arm.

In some examples, once the distal tip 419 of the guidewire 18 is received within the right pulmonary artery 14, the crossing catheter 416, or a different dilating catheter, may then be advanced distally through the outer steerable catheter 414, over the guidewire 418, and through the puncture created in the wall of the superior vena cava 12 and the wall of the right pulmonary artery 14. This may dilate the puncture into a larger opening capable of receiving the deployment system 412 and the shunt support structure 420 thereof. In other examples, once the puncturing tip 417 extends into the right pulmonary artery 14, the guidewire 418 may be advanced distally through the outer steerable catheter 414, and the puncturing tip 417 of the crossing catheter 416 into the right pulmonary artery 14.

Subsequently, the crossing catheter 416 may be withdrawn through the outer steerable catheter 414 and the first lumen 402 of the catheter sheath 401, and deployment system 412 may then be advanced into the first lumen 402, over the guidewire 418, to deliver the shunt support structure 420 partially into (e.g., about 50 percent or halfway through) the puncture created between the superior vena cava 12 and the right pulmonary artery 14. In an alternative example, the crossing catheter 416, the outer steerable catheter 414, and the guidewire 418 may each be removed, and a different guidewire, such as the delivery guidewire 111 (FIG. 8) sized and shaped for guiding the deployment system 412 to a target location, may then be inserted into the right pulmonary artery 14 via advancement through the first lumen 402 and the puncture between the superior vena cava 12 and the right pulmonary artery 14 before or after the outer steerable catheter 414 is removed.

Next, the shunt support structure 420 may be radially expanded, such as via a balloon located within the deployment system 412 and/or self-expansion of the shunt support structure 420, such as discussed previously discussed with respect to other delivery catheters of the present disclosure, to thereby create a shunt passage between the between the superior vena cava 12 and the right pulmonary artery 14. The deployment system 412 may then be retracted or withdraw, first from within the shunt support structure 420 and then proximally through the first lumen 402. Finally, the anchoring system 432 may be radially compressed or deflated, and the catheter system 400 including the catheter sheath 401 may be removed from the patient through its original access point.

While the above method is generally discussed in the context of creating a shunt or shunt passage from the superior vena cava 12 into the right pulmonary artery 14, it is to be appreciated that the catheter system 400, and the various additional systems or devices discussed below with respect to the FIGS. 61-70, may be also used to create a shunt or shunt passage between the right pulmonary artery 14 and the superior vena cava 12, or between other adjacently located blood vessels or anatomic features of a patient. In view of all the above, the catheter system 400, and the various systems or devices discussed below with respect to FIGS. 61-70, can provide benefits to both patients and physicians.

For example, the imaging capabilities of the imaging catheter 426 may enable a physician to reduce the total number of fluoroscopic steps or operations used during a similar procedure, such as first by eliminating the need to first position and operate a C-arm to guide the catheter system 400 into a location proximal, or adjacent, to a target puncturing location. This may make the insertion process of a shunt creation system simpler for a physician, as well as helping to reduce the time required to guide a shunt creation system to a target location within a patient. Second, the catheter system 400 may enable a physician to further reduce the number of fluoroscopic steps (e.g., the amount of C-arm use) by providing the ability to confirm a final position of the catheter system 400 at a puncturing location before puncturing operations begin. This may help to eliminate the use of, or reduce the amount of, contrast dye introduced into a patient for fluoroscopic imaging purposes, which may in turn eliminate or reduce procedural complications such as allergic reactions.

Additionally, in some examples, the imaging capabilities provided by the imaging catheter 426 may eliminate the need to surgically access both vessels through which a shunt passage is to be created, such as by enabling a physician to position the catheter system 400 to a target vessel without the use of a targeting device or a catheter first inserted into the target vessel via a second access site. This may make the insertion process of a puncturing system less traumatic process for a patient, as well as help to further reduce procedural complications and the time required to guide shunt creation system to a target location within a patient.

FIG. 61 is a view of a catheter system 446 in the superior vena cava 12. The catheter system 446 may be similar to the catheter system 400, except in that the catheter system 446 includes the deployment system 448 as an alternative to the deployment system 412. The deployment system 448 may, like the deployment system 412, include any of the elements or features of the various delivery catheters capable of deploying shunt support structures previously discussed in this disclosure above. However, in contrast to the deployment system 412, the deployment system 448 may include a suture wrap 450.

The suture wrap 450 may extend radially around a circumference of a shunt support structure 452 located on the deployment system 448. The suture wrap 450 may include any number of circumferential wraps or coils about the shunt support structure 452, such as but not limited to, two, three, four, five, six, seven, eight, nine, ten, or greater numbers or wraps or coils. In some examples, the suture wrap 450 may alternatively be a braided or a woven suture, such as to form a mesh covering for the shunt support structure 452. The suture wrap 450 may be made from a material adapted to break, release, or otherwise sever upon expansion or inflation of an expandable balloon located within the shunt support structure 452 of the deployment system 448. The suture wrap 450 may help a physician to avoid unintentionally moving or disturbing one or more vessel walls, such as often caused by the retraction or removal of overlying protective sheaths or capsules covering various shunt support structures of delivery catheters.

In some examples, the catheter system 400 may additionally include one or more radiopaque markers, to help a physician visualize any of the distal portion 406 of the catheter sheath 401, the distal portion 415 of the outer steerable catheter 414, or the crossing catheter 416 (FIGS. 59-60) during fluoroscopic imaging, such as provided by a C-arm or other devices, or echogenic imaging via the imaging catheter 426. In some examples, such as shown in FIG. 61, the catheter system 400 may include a first marker 438 located near the distal end 408 of the catheter sheath 401, a pair of markers 440 located on one side of the distal portion 415 of the outer steerable catheter 414, and a second marker 442 located on an opposite side of the distal portion 415 (e.g., 180 degrees circumferentially offset from the pair of markers 440). In one or more such examples, a physician may be able to determine an amount of deflection of the outer steerable catheter 414 or determine in which direction the distal portion 415 is flexing, based upon a visual comparison of the relative positions of any of the first marker 438, the pair of markers 440, and the second marker 442.

In additional examples, the first marker 438, the pair of markers 440, the second marker 442, or other markers of the catheter system 400, may alternatively be echogenic markers, such as to enable a physician to further reduce, or eliminate, the use of fluoroscopy during various steps of a shunt creation procedure, such as by utilizing, for example, the echocardiography imaging provided by the imaging catheter 426 to monitor the position of the distal portion 406 of the catheter sheath 401, the distal portion 415 of the outer steerable catheter 414, or the crossing catheter 416 (FIGS. 59-60) without relying on fluoroscopic imaging. In further examples, the catheter system 400 may include both radiopaque markers and echogenic makers, such as located in similar or different positions relative to any of the first marker 438, the pair of markers 440, or the second marker 442.

In still further examples, the outer steerable catheter 414, or any other steerable catheter of the present disclosure, may be configured to apply suction through one or more suction lumen extending therein and connected to a suction generated apparatus at a proximal end thereof. In such examples, said suction may anchor any steerable component through which suction is applied to a wall of the superior vena cava 12, or any other blood vessel in which the catheter system 400 may be positioned to thereby brace the steerable component against deflection in a direction opposite the curvature thereof, and thereby help to allow, for example, the deployment system 448, or the guidewire 418, or any other device passable therethrough to be directed in a more consistent and predictable manner. In a further example, said be used as an alternative anchoring means to the anchoring system 432.

In additional examples, the guidewire 418, the outer steerable catheter 414, the crossing catheter 416 (FIGS. 59-60), the deployment system 448, or any other puncturing or dilating component of the catheter system 400 may be cryogenically cooled, such as by including gas or liquid lumens therein in fluid communication with a gas or fluid supply, and/or or tubes or lumens in fluid communication with a Joule-Thompson orifice, valve, or porous plug near a puncturing or dilating tip thereof (e.g., of the crossing catheter 416 or the distal portion 429 of the deployment system 412). In such examples, the puncturing or dilating tips of various components of the catheter system 400 can be cryogenically cooled during a shunt creation procedure. This may enable, for example, the distal end 415 of the outer steerable catheter 414 to, when cryogenically cooled or frozen, adhere or otherwise stick to a vessel wall, and thereby help to allow the crossing catheter 416, the guidewire 418, or the deployment system, 448 to be directed therethrough in a more consistent and predictable manner.

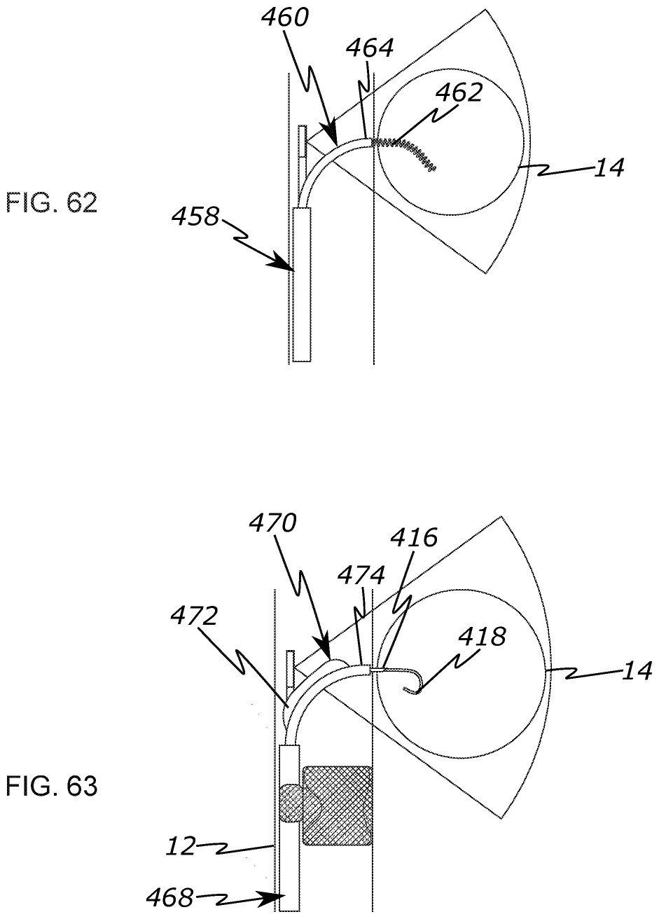

FIG. 62 is a view of a catheter system 458 in the superior vena cava 12. The catheter system 458 may be similar to the catheter system 400, except in that the catheter system 446 includes a puncturing system 460 as an alternative to the deployment system 412. The puncturing system 460, like the puncturing system 410, may include various elements or features of any of the puncturing systems, puncturing guidewires, crossing or steerable catheters, or other devices discussed in this disclosure. However, in contrast to the puncturing system 410, the puncturing system 460 may include a guidewire including a helical tip 462.

The helical tip 462 may form a helical, corkscrew, or spiral shape, but may also form any curving or repeating profile that may be capable of penetrating tissue or a vessel wall while, for example, undergoing rotation. The helical tip 462 may optionally be RF enabled, such as by including electrodes and various features of the RF enabled puncturing guidewires or crossing catheters previously discussed in the present disclosure above. In some examples, the helical tip 462 may be made from a shape memory material, such as, but not limited to, Nitinol, such to enable the helical tip 462 to be advanced through an unmodified steering or crossing catheter and form its helical shape only upon distally advancement from the steerable catheter, such as the crossing catheter 416 (FIGS. 58-60) or the outer steerable catheter 414 (FIGS. 58-60).

In other examples, the helical tip 462 may permanently form its helical shape, and the puncturing system 460 may include a steerable catheter 464 that is internally sized and shaped to enable the helical tip 462 to be advanced and retracted therethrough, such as without changing shape. In still further examples, the helical tip 462 may alternatively be realized via a similar helical shaping of the puncturing tip 417 of the crossing catheter 416, and, in such an example, the helical tip 462 may accordingly be a hollow tip configured to enable a standard or non-helical guidewire to be advanced there through. In an addition example, the catheter system 458 may include, or be connected to, a means for rotating the helical tip 462 during advancement or penetration of a vessel wall therewith. The helical tip may, after penetration with, and by virtue its coiled or spiral shape, anchor two adjacent blood vessels to one another to prevent subsequent relative movement therebetween. This may help a physician to avoid unintentionally moving one or more vessel walls away from the other, such as caused by the movement of a dilating cross catheter or a shunt delivery catheter over the guidewire and through a puncture between the vessel walls.

FIG. 63 is a view of a catheter system 468 in a superior vena cava 12. The catheter system 468 may be similar to the catheter system 400, except in that the catheter system 468 includes a deflectable catheter 470, such as alternatively to the outer steerable catheter 414. The deflectable catheter 470 may be similar to, and include any of various features or aspects of, the outer steerable catheter 414, at least in that it may be selectively bent or curved between a first position or configuration extending parallel to the central axis A1 into a second position or configuration (shown in FIG. 63) that may be angled between at about 90 degrees relative to the central axis A1, such as to direct the crossing catheter 416 and/or the guidewire 418 towards a wall of the vessel superior vena cava 12 and the right pulmonary artery 14. However, in contrast to the outer steerable catheter 414, which may, for example, be selectively deflected or curved via actuation of a pull wire, the deflectable catheter 470 may be selectively steered or curved based upon an amount of inflation of a deflection balloon 472 located along a distal portion 474 of the deflectable catheter 470.

The deflectable catheter 470 may include one or more inflation lumens extending therein for communicatively connecting the deflection balloon 472 to a fluid supply to enable the at least one balloon to be inflated. In some examples, the deflection balloon 472 may be similar to the balloon 246 or the balloon 256 discussed with respect to FIGS. 33-36, in which case the deflection balloon 472 may be configured to contact a wall of the superior vena cava 12 opposite the right pulmonary artery 14, or a wall of any vessel in which the catheter system 468 may be located that is opposite a puncture or stent creation location. The deflection balloon 472 may also function to, with or without contact with a wall of a vessel, help to brace the distal portion 474 in a direction opposite of the deflection or curvature thereof, and thereby help to allow the crossing catheter 416 or the guidewire 418 to be directed in a more consistent and predictable manner.

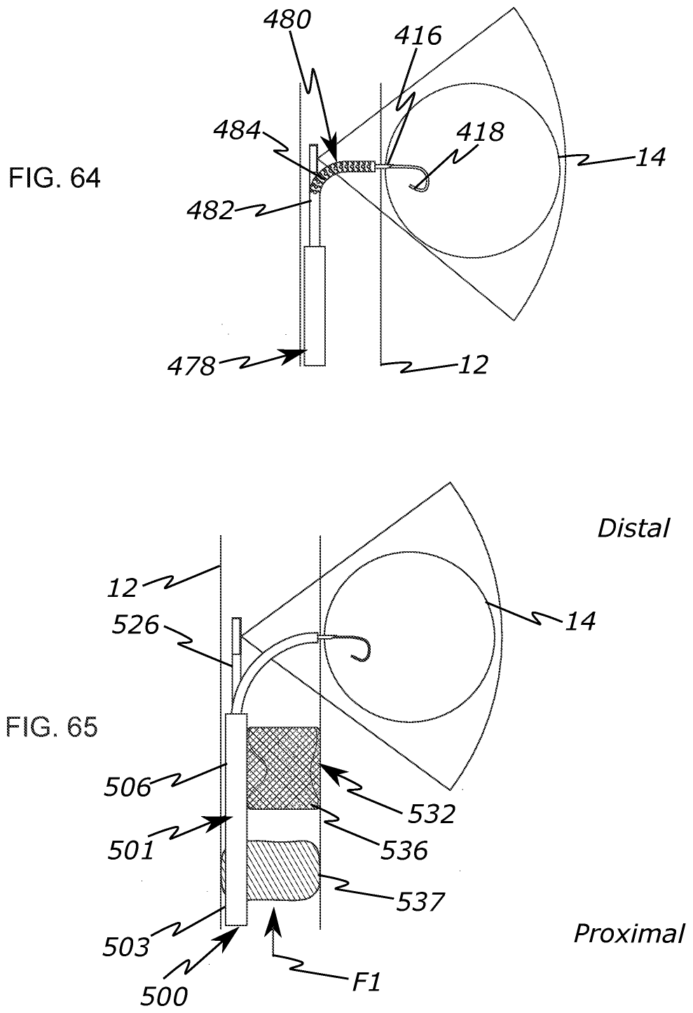

FIG. 64 is a view of a catheter system 478 in the superior vena cava 12. The catheter system 478 may be similar to the catheter system 400, except in that the catheter system 478 includes a reinforced steerable catheter 480, such as alternatively to the outer steerable catheter 414. The reinforced steerable catheter 480 may be similar to, and may include any of various features or aspects of, the outer steerable catheter 414, at least in that it may be adapted to be selectively bend or curve in only one direction between a first position or configuration, extending parallel to the central axis A1, into a second position or configuration (shown in FIG. 64), extending about 90 degrees relative to the central axis A1, such as to direct the crossing catheter 416 and/or the guidewire 418 towards a wall of superior vena cava 12 and the right pulmonary artery 14.

However, in contrast to the outer steerable catheter 414, the reinforced steerable catheter 480 may include a distal portion 482 including a reinforcement region 484 made from a relatively rigid material, such as, but not limited to, laser-cut, machined, or stamped stainless steel or titanium. In some examples, the reinforcement region 484 may be realized in the form of a slotted, segmented, jointed, or otherwise articulatable hypotube configured to bend or curve only in a single direction or plane, as well as to receive and guide the crossing catheter 416 and/or the guidewire 418. In other examples, the reinforcement region 484 of the distal portion 482 may alternatively be realized in the form of a distal portion of a steerable catheter, such as similar to any of the other steerable or deflectable catheters or devices of the present disclosure previously described above, having a slotted, segmented, jointed, or otherwise bendable hypotube positioned thereover.

In view of the above, the reinforcement region 284 may increase the ability of the reinforced steerable catheter 480 to flex or deflect only within a single plane, such as to guide various puncturing or delivery catheters therethrough, while more effectively resisting other forces that may cause undesirable deflection or movement. For example, the reinforcement region 284 may help the to resist forces acting in a direction opposite the direction in which the reinforcement catheter 280 is configured to bend or curve, as well as help prevent deflection of the distal portion 482 in other planes or directions, such as orthogonal to the plane in which the reinforcement catheter 280 is configured to bend or curve. This may help to allow, for example, the crossing catheter 416 or the guidewire 418 to be directed in a more consistent and predictable manner. Further, in one such example, this may enable the reinforced steerable catheter 480 to be used without the anchoring system 432, without a bracing element such as the one or more balloon 256, the wire frame or structure 264, or the one or more arms 272 shown in FIGS. 35-37.

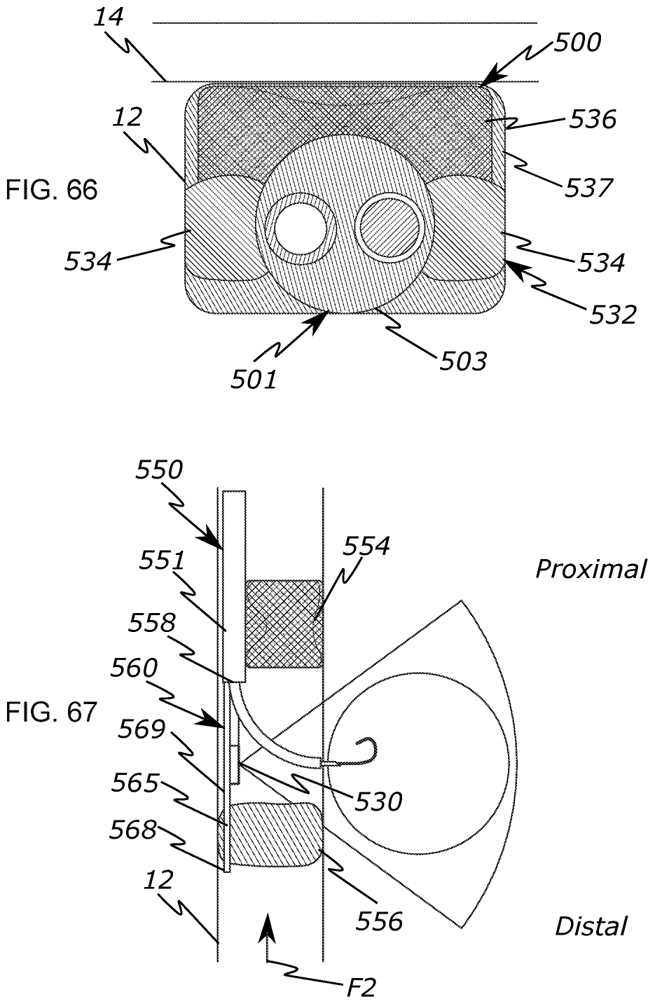

FIG. 65 is a view of a catheter system 500 in the superior vena cava 12. FIG. 66 is a cross-section of the catheter system 500 of FIG. 65 in the superior vena cava 12. Also shown in FIG. 65 are orientation indicators Proximal and Distal, relating to relative positions along a catheter sheath 501. FIGS. 65-66 are discussed below concurrently. The catheter system 500 may be similar to the catheter system 400 previously discussed above respect to FIGS. 58-60, except in that the catheter sheath 501 of the catheter system 500 includes a proximal sealing element 537. The proximal sealing element 537 may be located on the distal portion 506 (FIG. 65) of the catheter sheath 501, such as, in some examples, in location proximal to an anchoring system 532.

The anchoring system 532 may be similar to the anchoring system 432 (FIGS. 58-60), and, in some examples, may include one or more balloons 534 and a secondary element 536. The proximal sealing element 537 may be similar to any of the various expandable elements or devices of the anchoring system 532, at least in that the proximal sealing element 537 may be a component configured to expand radially outwardly from an outer surface 503 of the catheter sheath 501 to engage a vessel wall, such as of the superior vena cava 12. However, in contrast to the various expandable components of the anchoring system 532, the proximal sealing element 537 is adapted to, when expanded, fill and seal the vessel in which it is positioned to prevent blood from passing thereby.

In some examples, such as shown in FIGS. 65-66, the proximal sealing element 537 may be realized in the form of a single sealing balloon, extending radially about the outer surface 503 of the catheter sheath 501, and adapted to engage an entire circumference of a wall of the superior vena cava 12, or any other vessel in which the catheter system 500 may be positioned. In other examples, the proximal sealing element 537 may be realized in the form of a plurality of balloons, such as, but not limited to, two, three, four, or five balloons, collectively extending circumferentially around the outer surface 503 of the catheter sheath 501, and adapted to engage an entire circumference of a wall of the superior vena cava 12, or any other vessel in which the catheter system 500 may be positioned.

In any of the above examples, the catheter sheath 501 may include one or more separate (e.g., independent from the anchoring system 532) inflation lumens for communicatively connecting the proximal sealing element 537 to a fluid supply, to enable the proximal sealing element 537 to be inflated or deflated independently of one or more balloons of the anchoring system 532. In further examples, the proximal sealing element 537 may alternatively include, or be realized in the form of, one or more mechanical devices such as including, but not limited to, one or more arms, laser cut or welded expandable wireframes or wire frameworks, rings, braided meshes or braided segments, that may each include non-porous membranes, shields, or other sealing materials or layers, which may be radially expanded to engage an entire circumference of a wall of the superior vena cava 12, or any other vessel in which the catheter system 500 may be positioned.

In one example use, the catheter system 500 may be selected for a shunt creation operation in which the catheter sheath 501 will experience blood flow there past in a proximal to distal direction (indicated by arrow F1), such as, but not limited to, in a procedure where the superior vena cava 12 is accessed from a location above the heart, such via a jugular vein, or where the right pulmonary artery is accessed from a location below the heart, such as via a femoral vein. The catheter system 500 may then be advanced into, and anchored at, a location within the superior vena cava 12 that is adjacent or proximal to the right pulmonary artery 14, such as by using the imaging catheter 526 in a manner similar to as described with respect to the imaging catheter 426 (FIGS. 58-60) above. Next, before any puncturing operations occur, the proximal sealing element 537 may be first be expanded to temporarily cut off blood flow there past.

This may significantly reduce blood pressure at, or near, the puncture location, which, may in turn reduce the risk of internal bleeding between the superior vena cava 12 and the right pulmonary artery 14 or vice versa when, for example, a puncture therebetween is created, when the puncture is dilated, or during the time in which a shunt support structure is subsequently expanded within the puncture. Subsequently, after the shunt support structure, such as the shunt support structure 420 (FIG. 59), has been expanded between the superior vena cava 12 and the right pulmonary artery 14 to establish a shunt passage therebetween, the proximal sealing element 537 may be deflated or retracted.

FIG. 67 is a view of a catheter system 550 in the superior vena cava 12. Also shown in FIG. 67 are orientation indicators Proximal and Distal, relating to relative positions along a catheter sheath 551. The catheter system 550 may be similar to the catheter system 400 previously discussed above respect to FIGS. 58-60, except in that the catheter system 550 includes a distal sealing element 556. The distal sealing element 556 may be spaced distally apart from a distal end 558 of the catheter sheath 551, and, in some examples, an anchoring system 554. The anchoring system 554 may be similar to the anchoring system 432 (FIGS. 58-60), at least in that the distal sealing element 556 may be representative of one or more components configured to expand to contact and engage a vessel wall, such as of the superior vena cava 12 or any other vessel in which the catheter system 550 may be located, to help prevent relative movement therebetween.

However, in contrast to the various expandable components of the anchoring system 532, the distal sealing element 556 is adapted to, when expanded, fill and seal the vessel in which it is positioned to prevent blood from passing thereby. The distal sealing element 556 may be spaced, or otherwise advanced, distally apart from the distal end 558 of a catheter sheath 551 by a variety of different means. In one example, such as shown in FIG. 67, the catheter system 550 may include a sealing catheter 560 include the distal sealing element 556. In such an example, the catheter sheath 551 may, for example, may define a third lumen (not shown) sized and shaped to receive the sealing catheter 560 therethrough. Such a third lumen may extend parallel to, and laterally offset from, a first lumen (e.g., the first lumen 402) and a second lumen (e.g., the second lumen 404) within the catheter sheath 551, to enable the sealing catheter 560 to be advanced distally beyond the distal end 558 in an orientation parallel to the imaging head 530.

In one example, such as shown in FIG. 67, the sealing catheter 560 may be a custom, or proprietary, balloon catheter including an inflation lumen extending therein connected to a fluid supply for inflating the distal sealing element 556, and an outer shaft 565 having the distal sealing element 556 located about a distal end 568 thereof. In other examples, the sealing catheter 560 may be representative of any commercially available balloon catheter that may be insertable through the catheter sheath 501, via the third lumen, and inflate in a distal location to seal the blood vessel in which the catheter system 550 is positioned. In further examples, the sealing catheter 560 may be a preloaded, or a non-removable, component of the distal end 558 that is distally advanceable therefrom to deploy the distal sealing element 556.

In some examples, such as shown in FIG. 67, the distal sealing element 556 may be realized in the form of a single sealing balloon, extending radially around an outer surface 569 of the outer shaft 565 of the sealing catheter 560, adapted to engage an entire circumference of a wall of the superior vena cava 12, or any other vessel in which the catheter system 550 may be positioned. In other examples, the distal sealing element 556 may be realized in the form of a plurality of balloons, such as, but not limited to, two, three, four, or five balloons, collectively extending circumferentially around the outer surface 569 of the sealing catheter 560, and adapted to engage an entire circumference of a wall of the superior vena cava 12, or any other vessel in which the catheter system 550 may be positioned.

In any of the above examples, the sealing catheter 560 may also include one or more separate (e.g., independent from the anchoring system 554) inflation lumens for communicatively connecting the distal sealing element 556 to a fluid supply, to thereby enable the distal sealing element 556 to be inflated or deflated independently of one or more balloons of the anchoring system 554. In further examples, the proximal sealing element 556 may alternatively include, or be realized in the form of, one or more mechanical devices such as including, but not limited to, one or more arms, laser cut or welded expandable wireframes or wire frameworks, rings, braided meshes or braided segments, that may each include non-porous membranes, shields, or other sealing materials or layers, which may be radially expanded to engage an entire circumference of a wall of the superior vena cava 12, or any other vessel in which the catheter system 550 may be positioned.

In one example use, the catheter system 550 may be selected for a shunt passage creation operation where the catheter sheath 551 will experience blood flow there past in a distal to proximal direction (indicated by arrow F2) along the catheter sheath 551, such as, but not limited to, in a procedure where the superior vena cava 12 is accessed from a location below the heart, such via a femoral vein, or where the right pulmonary artery is accessed from a location above the heart, such as via a jugular vein. As may be appreciated in view of the above, this may enable the catheter system 550 to, when inserted into same access point of a patient in the same orientation, block a blood flow moving in a direction opposite to that of the catheter system 500 including the proximal sealing element 537 (FIGS. 55-56).

The catheter system 550 may then be advanced and guided to, and subsequently anchored at, a location within the superior vena cava 12 that is adjacent or proximal to the right pulmonary artery 14, such as by using the imaging head 530 in method similar to as described with respect to the imaging head 430 of the catheter system 400 (FIGS. 58-60). Next, before any puncturing operations occur, the distal sealing element 556 may first be advanced distally beyond the distal end 558, the imaging head 430, and a selected puncture location. The distal sealing element 556 may then expanded to temporarily cut off blood flow there past.

This may significantly reduce blood pressure at, or near, the puncture location, which, may in turn reduce the risk of internal bleeding between the superior vena cava 12 and the right pulmonary artery 14 or vice versa when, for example, a puncture therebetween is created, when the puncture is dilated, or during the time in which a shunt support structure is subsequently expanded within the puncture. Subsequently, after the shunt support structure, such as the shunt support structure 420 (FIG. 59), has been expanded between the superior vena cava 12 and the right pulmonary artery 14 to establish a shunt passage therebetween, the distal sealing element 556 may be deflated.

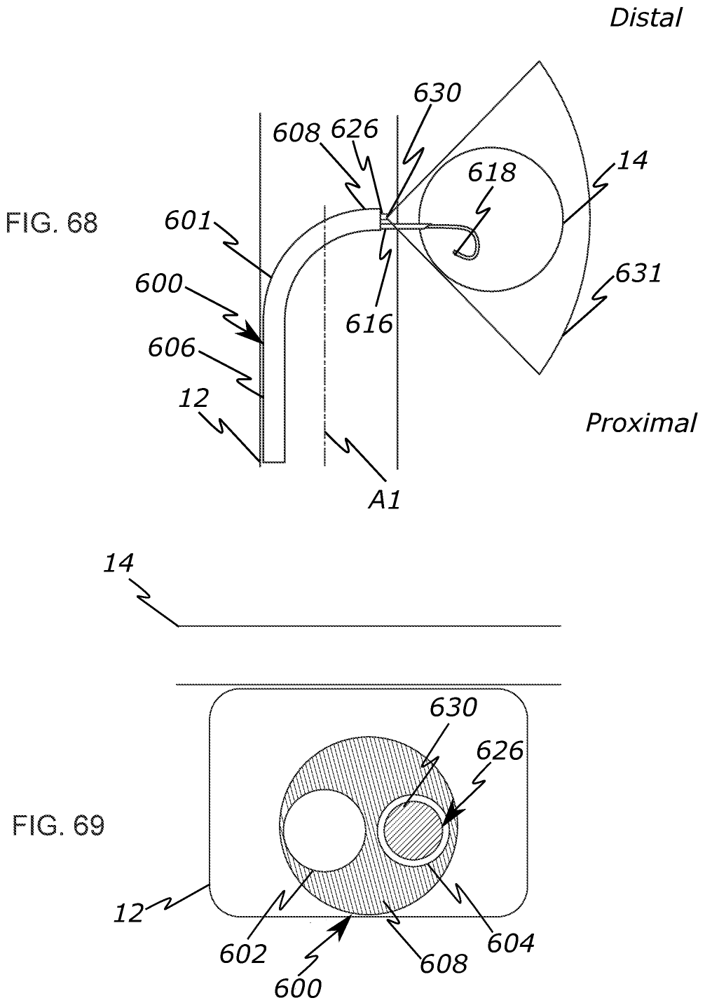

FIG. 68 is a view of a catheter assembly 600 in the superior vena cava 12. Also shown in FIG. 68 are orientation indicators Proximal and Distal, relating to relative positions along a catheter body 601, and a central axis A1 defined by the superior vena cava 12. The catheter body 601 may be similar to the catheter sheath 401, at least in the that the catheter body 601 may include a first lumen 602 (FIG. 69) adapted to receive a crossing catheter 616 and/or a guidewire 618, each of which may be similar to the crossing catheter 416 or the guidewire 418 (FIGS. 58-60), and a second lumen 604 (FIG. 69) adapted to receive an imaging system 626.

However, first, in contrast to the catheter sheath 401, the catheter body 601 may be, or may function as, a steerable or selectively deflectable catheter configured for directing the crossing catheter 616 and/or the guidewire 618 toward a vessel wall. For example, the catheter body 601 may be selectively bent or curved between a first position or configuration (shown in FIG. 69) extending parallel to the central axis A1 (FIG. 68) into a second position or configuration (shown in FIG. 68) that may be, but is not limited to, angled at about 90 degrees relative to the central axis A1, via actuation of a pull wire and/or user controls on a proximal end thereof. The imaging system 626 may, in some examples, be similar to the imaging catheter 426, at least in that the imaging system 626 may include an imaging head 630 adapted to perform imaging techniques or operations including, but not limited to, phased array, or radial or rotational, Intracardiac Echocardiography (hereinafter “ICE”) imaging, or alternatively Intravascular Ultrasound (“UVUS”) imaging.

Second, in further contrast to the imaging catheter 426, the imaging system 626 may be configured to perform imaging operations when the imaging head 630 is located in a position perpendicular to the central axis A1, which, as may be appreciated, is necessitated by the curvature of the distal end 608 of the catheter body 601 toward a wall of the superior vena cava 12 and the right pulmonary artery 14. In some examples, the imaging system 626 may be an integral imaging device integrated into the distal end 608 of the catheter body 601.

In some additional examples, the distal portion 606 or the distal end 608 of the catheter body 601 may be reinforced in a manner similar to the reinforcement region 484 of the reinforced steerable catheter 480 as described with respect to FIG. 64. In further examples, the catheter body 601 may also include an anchoring system, such as the anchoring system 432, to help prevent relative movement of the catheter body 601 within a vessel during puncturing or shunt delivery operations, or the proximal sealing element 537 (FIGS. 65-66), to help lower the risk of internal between vessels during puncturing or shunt delivery operations.

In view of the above, the catheter assembly 600 may be used in a method similar those described with respect to the catheter system 400. For example, the catheter body 601 may be inserted into a patient via an access point, and then advanced distally into the patient, such as until the distal end 608 of the catheter body 601 enters a desired vessel, such as the superior vena cava 12. The position of the catheter assembly 600 may then be tuned or adjusted using the imaging system 626 to position the distal end 408 in a location proximal to a target vessel, such as adjacently to the right pulmonary artery 14. For example, the imaging system 626 may first be inserted into, and advanced distally through, the second lumen 604 of the catheter body 601, such as until the imaging head 630 enters the superior vena cava 12 through the distal end 608.

Next, using the imaging system 626, a physician may determine (e.g., visualize on a screen within a field of view 631) a relative position of imaging head 430 relative to the pulmonary artery 14 or other anatomical features located externally nearby the superior vena cava 12, such as, in some examples, by deflecting or curved the distal end 608 of the catheter body 601 about 90 degrees relative to the central axis A1 so that the field of view 631 is facing toward, or otherwise encompasses when aligned with, the right pulmonary artery. In response, the physician may advance, or retract, the catheter body 601 within the superior vena cava 12, such as until the physician is satisfied that the imaging head 630 is positioned at a height about equal to a center of the right pulmonary artery 14.

In one example, this may be when the right pulmonary artery 14 is centered within the field of view 631 of the imaging head 630, such as in a phased array configuration of the imaging head 630. In other example, this may be when the right pulmonary artery 14 is visible to the physician, such as in a radial or rotational configuration of the imaging head 630. In such an example, the distal end 608 may extend parallel to the central axis A1 during imaging operations, after which the distal end 608 may be deflected or curved toward the right pulmonary artery 14 or other vessels. The catheter assembly 600 may then optionally be anchored within the superior vena cava 12, such to help prevent relative movement between the catheter body 601 and the superior vena cava 12, and thereby, provide for increased stability and predictability during the subsequent advancement of the crossing catheter 616, the guidewire 618, or various other devices for performing puncturing or shunt delivery operations. Subsequently, said puncturing or shunt delivery operations may be carried out in a manner similar to as discussed with respect to various examples previously disclosed above.

FIG. 70 illustrates a view of a catheter assembly 700 in a superior vena cava 12. The catheter assembly 700 may be similar to the catheter assembly 600, except in that the catheter body 701 of the catheter assembly 700 may form an S-curve shape or profile. In such examples, the catheter body 701 may be sized and shaped to repeatedly engage opposite walls of the superior vena cava 12, or any other vessel in which the catheter assembly 700 may be positioned, through a plurality of contact points 702. As may be appreciated, the plurality of contact points 702 may be based on the number of curves, zigzags, or directional changes that the catheter body 701 includes. In one example, such as shown in FIG. 70, the plurality of contact points 702 may include three contact points. In other examples, the plurality of contact points 702 may include two, four, five, size, seven, eight, nine, ten, or still other numbers of contact points.

In an example use, the catheter body 701, at the plurality of contact points 702, may frictionally engage a wall of a vessel, such as the superior vena cava 12, to brace a distal portion 706 of the catheter body 701 against lateral forces during, for example, advancement of the crossing catheter 616, the guidewire 618, or a delivery catheter therethrough into the right pulmonary artery 14. Moreover, the frictional engagement between the plurality of contact points 702 and the wall of the vessel may, in some examples, help the catheter body 701 resist axial movement within a vessel without the use of an expandable or an inflatable anchoring system.

FIG. 71 illustrates a view of a catheter assembly 800 in a superior vena cava 12. The catheter assembly 800 may be similar to the catheter assembly 600, except in that a catheter body 801 of the catheter assembly 800 may form a helical, spiral, or a corkscrew shape or profile. In such examples, the catheter body 801 may be sized and shaped to continuously engage a wall of the superior vena cava 12, or any other vessel in which the catheter assembly 800 is positioned, with a helical outer surface 804 thereof. The helical outer surface 804 may include or may be defined by, for example, but not limited to, one, two, three, four, five, six, seven, eight, or other numbers of individual or complete circumferential coils. In one example, such as shown in FIG. 70, the helical outer surface 804 may include by four circumferential coils.

In an example use, the catheter body 801 may, through the helical outer surface 804, frictionally engage a wall of a vessel, such as the superior vena cava 12, to brace a distal portion 806 of the catheter body 801 against lateral forces during, for example, advancement of the crossing catheter 616, the guidewire 618, or delivery catheter therethrough into the right pulmonary artery 14. Moreover, the frictional engagement between the helical outer surface 804 wall of the vessel may help the catheter body 801 to resist axial movement within a vessel without the use of an expandable or an inflatable anchoring system.

In some further examples, any non-imaging steerable catheter, catheter system, or catheter assembly of the present disclosure described above may be guided into the patient using imaging techniques including, but not limited to, phased array, or radial or rotational, Intracardiac Echocardiography (hereinafter “ICE”) imaging, or alternatively, Intravascular Ultrasound (“UVUS”) imaging. First, the non-imaging steerable catheter, catheter system, or catheter assembly may be inserted into a patient via an access point, such as in a femoral vein in a leg of a patient as described with respect to FIGS. 4-5, and then be advanced distally into the patient, such as until a distal portion thereof enters a desired vessel, such as the superior vena cava 12.

Next, a standalone or independent imaging catheter may first be inserted from a separate access point, such as in a jugular vein for the patient, and then be advanced distally into the patient until the imaging catheter enters the desired vessel, such as the superior vena cava. Subsequently, the imaging catheter may be moved towards the non-imaging steerable catheter, catheter system, or catheter assembly located in the desired vessel, such as until one or more magnets on opposite surfaces there, or other features such as an alignment funnel, contact and engage each other of prevent relative movement between an imaging head of the imaging catheter and the non-imaging steerable catheter, catheter system, or catheter assembly located in the desired vessel.

Finally, the position of the non-imaging steerable catheter, catheter system, or catheter assembly located in the desired vessel, may then be fined tuned or adjusted using the imaging system in order to position a reference point useable in vessel puncturing, such as a distal end of an outer steerable catheter, at a location proximal to a target vessel, such as adjacently to the right pulmonary artery.

In some examples, a method of creating a shunt passage using any of the systems or devices discussed with reference to FIGS. 58-70 above may include first advancing a targeting and/or grasping device in one of the vessels, followed by the advancing of a catheter system or assembly include a puncturing system or device into the other vessel, piercing both vessels via the puncturing system or device, and grasping o engaging a distal portion or distal tup of the puncturing device with targeting or grasping device.

For example, a target and/or grasping device, such as, but not limited to, any of the snare catheter 104, the snare catheter 180, the snare catheter 190, the snare catheter 191, the snare catheter 195, the snare catheter 200, the snare catheter 210, the snare catheter 220, or the snare catheter 204 previously discussed above, may be positioned within the right pulmonary artery 14 and the puncturing system 410, the crossing catheter 416, or the guidewire 418 may be inserted into the superior vena cava 12, or vice versa, through the catheter sheath 401 and/or the outer steerable catheter 414. In such examples, such as depending upon the snare catheter used, such a method may further include using magnets, radiofrequency energy, or any of feature to aid in targeting or alignment with the crossing catheter 416 or the guidewire 418 for targeting.

In some examples, a method of creating a shunt passage using any of the systems or devices discussed with reference to FIGS. 58-70 above may also include puncturing the superior vena cava 12 through a branch of a patient's pulmonary artery to create a shunt passage therethrough and the superior vena cava 12. In another example, a method of creating a shunt passage using any of the systems or devices discussed with reference to FIGS. 58-70 above may include accessing the right pulmonary artery 14 through a branch thereof, such as via initial advancement therethrough, before entering the main trunk of the right pulmonary artery 14.

This specification primarily discusses embodiments of the present invention with regard to a shunt connecting a right pulmonary artery to a superior vena cava. However, it is to be appreciated that shunts can be created at other locations for similar purposes using the systems, devices, and methods of this disclosure.

In one example, a main pulmonary artery (PA) is shunted to the right atrium or atrial appendage (RAA). In this method, a right-to-right shunt from a region of higher pressure in the PA is connected to a region of lower pressure in the RAA. Doing so utilizes the high compliance of the RAA to “absorb” additional volume received from the shunt since the RAA is a naturally compliant reservoir. An additional benefit may arise from the fact that the RAA and the main PA are both inside the pericardium and, therefore, would contain any leaks resulting as a complication of an improperly seated shunt. Another benefit may be that the risk of puncturing the aorta is minimized.

In another example, a connection made between a pulmonary artery (PA) and a pulmonary vein (PV) may be used to treat pulmonary hypertension or right heart failure/dysfunction. To reduce the total pulmonary vascular resistance and the afterload of the right ventricle, a shunt is created between a right pulmonary artery (RPA) and a right pulmonary vein (RPV). Alternatively, the shunt could be placed between a left pulmonary artery (LPA) and a left pulmonary vein (LPV).

In another example, a connection is created between a pulmonary artery (PA) and a left atrial appendage (LAA), in order to treat pulmonary hypertension, right heart failure/dysfunction, or atrial fibrillation, which reduces the total pulmonary vascular resistance and the afterload of the right ventricle. An added benefit to the reduced right ventricular afterload is the washout of the LAA in those patients that are at risk of stroke.

In yet another example, a shunt is created between a pulmonary vein (PV) and superior vena cava (SVC) to treat heart failure. This may particularly help treat elevated left atrial pressures causing fluid to back up in the lungs.

In yet another example, a plurality of shunts at different locations, such as any of the previously discussed locations can be used. For instance, there may be a benefit to placing an RPA-SVC shunt as well as an atrial shunt in certain populations. The RPA-SVC shunt would help reduce RV afterload and the LA shunt would help reduce PVR while keeping LA pressure and LV filling pressure low. To the same effect, there may be a benefit to the combination of the RPA-VC, intra-atrial, and arteriovenous peripheral shunt in certain patients.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

### Claim Bank

Clause 1. A method for creating a shunt, the method comprising: positioning a catheter sheath within a superior vena cava; inserting an imaging catheter into a lumen extending within the catheter sheath; advancing an imaging catheter out of a distal end of the catheter sheath; imaging a right pulmonary artery with the imaging catheter; anchoring the catheter sheath within the superior vena cava to prevent relative movement between the catheter sheath and the superior vena cava; advancing a puncturing guidewire of a puncturing system out of the distal end of the catheter sheath, through the superior vena cava, and into the right pulmonary artery; advancing a crossing catheter of the puncturing system of the distal end of the catheter sheath, through the superior vena cava, and into the right pulmonary artery; and expanding a shunt support structure within the superior vena cava and the right pulmonary artery to create a shunt passage.

Clause 2. The method of clause 1, wherein imaging the right pulmonary artery includes using phased array intracardiac echocardiography imaging, radial or rotational intracardiac echocardiography imaging, or intravascular ultrasound imaging provided by an imaging head of the imaging catheter.

Clause 3. The method of clause 2, wherein anchoring the catheter sheath includes inflating one or more anchoring balloons or expanding an anchoring wire framework on the catheter sheath to engage a vessel wall of the superior vena cava.

Clause 4. The method of clause 3, wherein positioning the catheter sheath within the superior vena cava includes first inserting the catheter sheath into a jugular vein, and wherein anchoring the catheter sheath includes inflating at least one balloon to block blood flow through the superior vena cava from a location distal to the one or more anchoring balloons or the anchoring wire framework.

Clause 5. The method of clause 3, wherein positioning the catheter sheath within the superior vena includes first inserting the catheter sheath into a jugular vein, and wherein anchoring the catheter sheath includes inflating at least one sealing balloon adapted to block blood flow through the superior vena cava from a location proximal to the one or more anchoring balloons or the anchoring wire framework.

Clause 6. The method of clause 1, wherein imaging the right pulmonary artery includes determining a location of the right pulmonary artery relative to the distal end of the catheter sheath within the superior vena cava.

Clause 7. The method of clause 1, wherein advancing the puncturing guidewire out of the distal end of the catheter sheath includes deflecting a distal tip of the puncturing guidewire about 90 degrees relative to the distal end of the catheter sheath.

Clause 8. The method of clause 7, wherein deflecting the distal tip of the puncturing guidewire about 90 degrees relative to the distal end of the catheter sheath includes inflating a balloon located along a distal region of a deflectable catheter of the puncturing system.

Clause 9. The method of clause 1, wherein advancing the puncturing guidewire driving a helically shaped distal tip of the puncturing guidewire through the right pulmonary artery and the superior vena cava to anchor the superior vena cava to the right pulmonary artery.

Clause 10. A method for creating a shunt, the method comprising: positioning a catheter sheath within a right pulmonary artery; inserting an imaging catheter into a lumen extending within the catheter sheath; advancing an imaging catheter out of a distal end of the catheter sheath; imaging a superior vena cava with the imaging catheter; anchoring the catheter sheath within the right pulmonary artery to prevent relative movement between the catheter sheath and the right pulmonary artery; advancing a puncturing guidewire of a puncturing system out of the distal end of the catheter sheath, through the right pulmonary artery, and into the superior vena cava; advancing a crossing catheter of the puncturing system out of the distal end of the catheter sheath, through the right pulmonary artery, and into the superior vena cava; and expanding a shunt support structure within the right pulmonary artery and the superior vena cava to create a shunt passage.

Clause 11. The method of clause 10, wherein imaging the superior vena cava includes using phased array intracardiac echocardiography imaging, radial or rotational intracardiac echocardiography imaging, or intravascular ultrasound imaging provided by an imaging head of the imaging catheter.

Clause 12. The method of clause 11, wherein anchoring the catheter sheath includes inflating one or more anchoring balloons or expanding an anchoring wire framework on the catheter sheath to engage a vessel wall of the superior vena cava.

Clause 13. The method of clause 12, wherein positioning the catheter sheath within the right pulmonary artery includes first inserting the catheter sheath into a jugular vein, and wherein anchoring the catheter sheath includes inflating at least one sealing balloon adapted to block blood flow through the right pulmonary artery from a location proximal to the one or more anchoring balloons or the anchoring wire framework.

Clause 14. The method of clause 12, wherein positioning the catheter sheath within the right pulmonary artery includes first inserting the catheter sheath into a femoral vein, and wherein anchoring the catheter sheath includes inflating at least one sealing balloon adapted to block blood flow through the right pulmonary artery from a location distal to the one or more anchoring balloons or the anchoring wire framework.

Clause 15. The method of clause 10, wherein imaging the superior vena cava includes determining a location of the superior vena cava relative to the distal end of the catheter sheath within the right pulmonary artery.

Clause 16. A method for creating a shunt, the method comprising: positioning a catheter sheath within a superior vena cava; inserting an imaging catheter into a lumen extending within the catheter sheath; advancing an imaging catheter out of a distal end of the catheter sheath; imaging a right pulmonary artery with the imaging catheter; anchoring the catheter sheath within the superior vena cava to prevent relative movement between the catheter sheath and the superior vena cava; advancing a puncturing crossing catheter of a puncturing system out of the distal end of the catheter sheath, through the superior vena cava, and into the right pulmonary artery; advancing guidewire of the puncturing system out of the catheter sheath, through the superior vena cava, and into the right pulmonary artery; and expanding a shunt support structure within the superior vena cava and the right pulmonary artery to create a shunt passage.

Clause 17. The method of clause 16, wherein imaging the right pulmonary artery includes using phased array intracardiac echocardiography imaging, radial or rotational intracardiac echocardiography imaging, or intravascular ultrasound imaging provided by an imaging head of the imaging catheter.

Clause 18. The method of clause 17, wherein positioning the catheter sheath within the superior vena cava includes first inserting the catheter sheath into a femoral vein, and wherein anchoring the catheter sheath includes inflating at least one sealing balloon adapted to block blood flow through the superior vena cava from a location proximal to the one or more anchoring balloons or the anchoring wire framework.

Clause 19. The method of clause 17, wherein positioning the catheter sheath within the superior vena includes first inserting the catheter sheath into a jugular vein, and wherein anchoring the catheter sheath includes inflating at least one sealing balloon adapted to block blood flow through the superior vena cava from a location distal to the one or more anchoring balloons or the anchoring wire framework.

Clause 20. The method of clause 16, wherein imaging the right pulmonary artery includes determining a location of the right pulmonary artery relative to the distal end of the catheter sheath within the superior vena cava.

Clause 21. A delivery catheter, comprising: an elongated catheter body; a shunt support structure radially compressed around a distal end of the elongated catheter body; a distal sleeve disposed over only a distal end of the shunt support structure; and, a proximal sleeve disposed over only a proximal end of the shunt support structure; wherein a middle portion of the shunt support structure is configured to remain uncovered when passing through a puncture through two vessels.

Clause 22. The delivery catheter of clause 21, wherein the distal sleeve and the proximal sleeve are conical.

Clause 23. The delivery catheter of clause 21, wherein one or more of the distal sleeve and the proximal sleeve are configured to slide away from the shunt support structure during delivery.

Clause 24. The delivery catheter of clause 22, wherein one or more of the distal sleeve and the proximal sleeve are configured be biased to a position partially covering the shunt support structure.

Clause 25. The delivery catheter of clause 22, wherein the one or more of the distal sleeve and the proximal sleeve include a releasable locking mechanism that unlocks the one or more distal sleeve and the proximal sleeve from being slidable relative to the elongated catheter body.

Clause 26. The delivery catheter of clause 21, wherein the one or more of the distal sleeve and the proximal sleeve are configured to at least partially tear so as to release the shunt support structure.

Clause 27. The delivery catheter of clause 21, further comprising an inflatable balloon disposed under the shunt support structure and a tacky coat disposed between the balloon and shunt support structure.

Clause 28. A delivery catheter, comprising: an elongated catheter body; a shunt support structure radially compressed around a distal end of the elongated catheter body; and, an RF electrode positioned on a distal tip of the elongated body and in communication with and configured to connect to an RF power supply.

Clause 29. The delivery catheter of clause 28, further comprising a sheath disposed over the shunt support structure and movable off of the shunt support structure; the sheath having a taper at a distal end of the sheath.

Clause 30. A puncturing guidewire, comprising: a wire body configured to delivery RF energy at a distal end of the wire body; a sheath disposed around a distal end of the wire body; wherein the sheath is configured to slide longitudinally away from the distal end of the wire body when the puncturing guidewire is pressed against tissue.

Clause 31. The puncturing guidewire of clause 30, wherein the sheath is biased to a position extending over the entire distal end of the wire body.

Clause 32. The puncturing guidewire of clause 30, wherein the sheath is configured to move proximally only a predetermined distance.

Clause 33. A puncturing guidewire, comprising: a wire body configured to delivery RF energy at a distal end of the wire body; a sheath disposed over the wire body; a handle connected to the wire body and the sheath; the handle comprising an position adjustment mechanism configured to move the sheath proximally relative to the wire body and limit proximal movement of the sheath so as to prevent the wire body from extending completely through two walls of a vessel.

Clause 34. A snare catheter, comprising: an elongated catheter body; an inflatable balloon located at a distal end of the elongated catheter body; and, one or more snare loops positioned within the balloon.

Clause 35. A snare catheter, comprising: an elongated catheter body; an inflatable balloon located at a distal end of the elongated catheter body; and, one or more snare loops positioned outside of and adjacent to the balloon.

Clause 36. The snare catheter of clause 15, wherein the balloon is composed of puncture resistant material.

Clause 37. A snare catheter, comprising: an elongated catheter body; one or more snare loops connected to a distal end of the elongated catheter body; a shield connected to a distal end of the elongated catheter body; the shield being positioned on one side of the one or more snare loops and configured to resist being pierced by a puncturing guidewire.

Clause 38. The snare catheter of clause 37, wherein the shield comprises a plurality of woven or braided wires, textile, a polyurethane sheet, or silicone.

Clause 39. The snare catheter of clause 37, wherein the shield has an oval shape, a planar shape, or a curve shape configured to conform to a vessel it is deployed within.

Clause 40. The snare catheter of clause 37, wherein the shield comprises an outer layer of electrically insulating material and an inner layer of electrically conductive material, and wherein the electrically conductive material is connected to an RF power supply so as to turn off the RF power supply upon contact with an RF puncturing guidewire with the electrically conductive material.

Clause 41. The snare catheter of clause 37, further comprising one ore move expandable balloons located proximally and/or distally of the shield.

Clause 42. The snare catheter of clause 41, further comprising a perfusion passage extending through the elongated catheter body and opening proximally and distally of the one or more balloons.

Clause 43. An RF catheter system comprising: a puncturing guidewire configured to puncture tissue with RF energy; a snare catheter having an elongated body; one or more RF electrodes connected at a distal end of the elongated body; and, an RF power source connected to the one or more RF electrodes and the puncturing guidewire.

Clause 44. A snare catheter system, comprising: an elongated catheter body; one or more snare loops connected to a distal end of the elongated catheter body; and, a magnetic field generating mechanism configured to create a magnetic field at a distal end of the elongated catheter body.

Clause 45. The snare catheter system of clause 44, further comprising a puncturing guidewire configured to sense or magnetically attract the magnetic field generating mechanism.

Clause 46. A steerable catheter, comprising: an elongated tubular catheter body configured to bend in a first direction via user controls on a proximal end of the catheter; and, a balloon positioned on one side of the catheter so as to expand in a direction opposite of the first direction.

Clause 47. A steerable catheter, comprising: an elongated tubular catheter body configured to bend in a first direction via user controls on a proximal end of the catheter; and, a wire frame member positioned on one side of the catheter so as to expand in a direction opposite of the first direction; wherein the wire frame comprises a loop or one or more arms.

Clause 48. A catheter system for creating a shunt between two vessels, comprising: an elongated catheter body having a passage extending therethrough and an aperture opening on a sidewall of the elongated catheter body and in communication with the passage.

Clause 49. The catheter system of clause 48, further comprising a puncturing guidewire configured to be positioned through the passage and out the aperture.

Clause 50. The catheter system of clause 48, wherein the aperture is located between about 1 to 2 cm from a distal end of the elongated catheter body.

Clause 51. The catheter system of clause 48, wherein the aperture has a diameter of about 0.1-0.5 cm.

Clause 52. The catheter system of clause 48, wherein the elongated catheter body further comprises an anchoring device near a distal end of the elongated catheter body; wherein the anchoring device comprises a balloon or a wire framework.

Clause 53. The catheter system of clause 48, further comprising one or more radiopaque markers positioned adjacent the aperture.

Clause 54. The catheter system of clause 48, further comprising one or more echogenic markers positioned adjacent the aperture.

Clause 55. The catheter system of clause 48, further comprising a first magnetic material positioned adjacent the aperture and further comprising a second elongated catheter body having a second magnetic material positioned near its distal end and configured to attract the first magnetic material.

Clause 56. A snare catheter, comprising: an elongated catheter body; and, a plurality of balloons connected at a distal end of the catheter body; wherein the plurality of balloons are spaced from each other to allow a puncturing guidewire to pass between them.

Clause 57. A method for creating a shunt, comprising: positioning one or more loops of a snare catheter within a right pulmonary artery; positioning a crossing catheter and a puncturing guidewire within a superior vena cava such that their distal ends are positioned near the one or more loops of the snare catheter; advancing the puncturing guidewire out of the superior vena cava and into the right pulmonary artery; and, advancing the crossing catheter from the superior vena cava to the right pulmonary artery.

Clause 58. The method of clause 57, wherein the snare catheter further comprises a shield positioned behind the one or more loops.

Clause 59. The method of clause 57, wherein positioning one or more loops of a snare catheter further comprising inflating a balloon on the snare catheter.

Clause 60. The method of clause 57, wherein advancing the puncturing guidewire out of the superior vena cava and into the right pulmonary artery further comprises limiting the longitudinal travel of the puncturing guidewire into the right pulmonary artery.

Clause 61. The method of clause 57, wherein advancing the puncturing guidewire out of the superior vena cava and into the right pulmonary artery further comprises contacting an electrode of the snare catheter.

Clause 62. A method for creating a shunt, comprising: positioning one or more loops of a snare catheter within a superior vena cava; positioning a first catheter and a puncturing guidewire within a right pulmonary artery such that their distal ends are positioned near the one or more loops of the snare catheter; advancing the puncturing guidewire out of the right pulmonary artery and into the superior vena cava; and, advancing a crossing catheter from the right pulmonary artery to the superior vena cava.

Clause 63. The method of clause 62, wherein advancing the puncturing guidewire out of the right pulmonary artery and into the superior vena cava further comprising advancing the puncturing guidewire out of an aperture in a sidewall of the first catheter.

Clause 64. The method of clause 62, wherein the snare catheter further comprises a shield positioned behind the one or more loops.

Clause 65. The method of clause 62, wherein positioning one or more loops of a snare catheter further comprising inflating a balloon on the snare catheter.

Clause 66. The method of clause 62, wherein advancing the puncturing guidewire out of the right pulmonary artery and into the superior vena cava further comprising limiting the longitudinal travel of the puncturing guidewire into the right pulmonary artery.

Clause 67. The method of clause 62, wherein advancing the puncturing guidewire out of the right pulmonary artery and into the superior vena cava further comprises contacting an electrode of the snare catheter.

What is claimed is:

1. A method for creating a shunt, the method comprising:
positioning a catheter sheath within a superior vena cava;
inserting an imaging catheter into a lumen extending within the catheter sheath;
advancing an imaging catheter out of a distal end of the catheter sheath;
imaging a right pulmonary artery with the imaging catheter;
anchoring the catheter sheath within the superior vena cava to prevent relative movement between the catheter sheath and the superior vena cava;
advancing a puncturing guidewire of a puncturing system out of the distal end of the catheter sheath, through the superior vena cava, and into the right pulmonary artery;
advancing a crossing catheter of the puncturing system of the distal end of the catheter sheath, through the superior vena cava, and into the right pulmonary artery; and
expanding a shunt support structure within the superior vena cava and the right pulmonary artery to create a shunt passage.

2. The method of claim 1, wherein imaging the right pulmonary artery includes using phased array intracardiac echocardiography imaging, radial or rotational intracardiac echocardiography imaging, or intravascular ultrasound imaging provided by an imaging head of the imaging catheter.

3. The method of claim 2, wherein anchoring the catheter sheath includes inflating one or more anchoring balloons or expanding an anchoring wire framework on the catheter sheath to engage a vessel wall of the superior vena cava.

4. The method of claim 3, wherein positioning the catheter sheath within the superior vena cava includes first inserting the catheter sheath into a jugular vein, and wherein anchoring the catheter sheath includes inflating at least one balloon to block blood flow through the superior vena cava from a location distal to the one or more anchoring balloons or the anchoring wire framework.

5. The method of claim 3, wherein positioning the catheter sheath within the superior vena includes first inserting the catheter sheath into a jugular vein, and wherein anchoring the catheter sheath includes inflating at least one sealing balloon adapted to block blood flow through the superior vena cava from a location proximal to the one or more anchoring balloons or the anchoring wire framework.

6. The method of claim 1, wherein imaging the right pulmonary artery includes determining a location of the right pulmonary artery relative to the distal end of the catheter sheath within the superior vena cava.

7. The method of claim 1, wherein advancing the puncturing guidewire out of the distal end of the catheter sheath includes deflecting a distal tip of the puncturing guidewire about 90 degrees relative to the distal end of the catheter sheath.

8. The method of claim 7, wherein deflecting the distal tip of the puncturing guidewire about 90 degrees relative to the distal end of the catheter sheath includes inflating a balloon located along a distal region of a deflectable catheter of the puncturing system.

9. The method of claim 1, wherein advancing the puncturing guidewire driving a helically shaped distal tip of the puncturing guidewire through the right pulmonary artery and the superior vena cava to anchor the superior vena cava to the right pulmonary artery.

10. A method for creating a shunt, the method comprising:
positioning a catheter sheath within a right pulmonary artery;
inserting an imaging catheter into a lumen extending within the catheter sheath;
advancing an imaging catheter out of a distal end of the catheter sheath;
imaging a superior vena cava with the imaging catheter;
anchoring the catheter sheath within the right pulmonary artery to prevent relative movement between the catheter sheath and the right pulmonary artery;
advancing a puncturing guidewire of a puncturing system out of the distal end of the catheter sheath, through the right pulmonary artery, and into the superior vena cava;
advancing a crossing catheter of the puncturing system out of the distal end of the catheter sheath, through the right pulmonary artery, and into the superior vena cava; and
expanding a shunt support structure within the right pulmonary artery and the superior vena cava to create a shunt passage.

11. The method of claim 10, wherein imaging the superior vena cava includes using phased array intracardiac echocardiography imaging, radial or rotational intracardiac echocardiography imaging, or intravascular ultrasound imaging provided by an imaging head of the imaging catheter.

12. The method of claim 11, wherein anchoring the catheter sheath includes inflating one or more anchoring balloons or expanding an anchoring wire framework on the catheter sheath to engage a vessel wall of the superior vena cava.

13. The method of claim 12, wherein positioning the catheter sheath within the right pulmonary artery includes first inserting the catheter sheath into a jugular vein, and wherein anchoring the catheter sheath includes inflating at least one sealing balloon adapted to block blood flow through the right pulmonary artery from a location proximal to the one or more anchoring balloons or the anchoring wire framework.

14. The method of claim 12, wherein positioning the catheter sheath within the right pulmonary artery includes first inserting the catheter sheath into a femoral vein, and wherein anchoring the catheter sheath includes inflating at least one sealing balloon adapted to block blood flow through the right pulmonary artery from a location distal to the one or more anchoring balloons or the anchoring wire framework.

15. The method of claim 10, wherein imaging the superior vena cava includes determining a location of the superior vena cava relative to the distal end of the catheter sheath within the right pulmonary artery.

16. A method for creating a shunt, the method comprising:
positioning a catheter sheath within a superior vena cava;
inserting an imaging catheter into a lumen extending within the catheter sheath;
advancing an imaging catheter out of a distal end of the catheter sheath;
imaging a right pulmonary artery with the imaging catheter;
anchoring the catheter sheath within the superior vena cava to prevent relative movement between the catheter sheath and the superior vena cava;
advancing a puncturing crossing catheter of a puncturing system out of the distal end of the catheter sheath, through the superior vena cava, and into the right pulmonary artery;
advancing guidewire of the puncturing system out of the catheter sheath, through the superior vena cava, and into the right pulmonary artery; and
expanding a shunt support structure within the superior vena cava and the right pulmonary artery to create a shunt passage.

17. The method of claim 16, wherein imaging the right pulmonary artery includes using phased array intracardiac echocardiography imaging, radial or rotational intracardiac echocardiography imaging, or intravascular ultrasound imaging provided by an imaging head of the imaging catheter.

18. The method of claim 17, wherein positioning the catheter sheath within the superior vena cava includes first inserting the catheter sheath into a femoral vein, and wherein anchoring the catheter sheath includes inflating at least one sealing balloon adapted to block blood flow through the superior vena cava from a location proximal to the distal end of the catheter sheath.

19. The method of claim 17, wherein positioning the catheter sheath within the superior vena includes first inserting the catheter sheath into a jugular vein, and wherein anchoring the catheter sheath includes inflating at least one sealing balloon adapted to block blood flow through the superior vena cava from a location distal to the distal end of the catheter sheath.

20. The method of claim 16, wherein imaging the right pulmonary artery includes determining a location of the right pulmonary artery relative to the distal end of the catheter sheath within the superior vena cava.
